(19) United States
(12) Patent Application Publication
(10) Pub. No.: US 2022/0403336 A1
(43) Pub. Date: Dec. 22, 2022

(54) NOVEL METHODS FOR PRODUCTION OF THERAPEUTIC MAMMALIAN CELLS AND CELL SPHERES AND COMPOSITIONS OF SAME

(71) Applicant: DiscGenics, Inc., Salt Lake City, UT (US)

(72) Inventors: Lara Silverman, Salt Lake City, UT (US); Daniel Rodriguez-Granrose, Salt Lake City, UT (US); Will Heaton, Salt Lake City, UT (US); Terry Tandeski, Salt Lake City, UT (US); Jeff Zurawski, Salt Lake City, UT (US); Niloofar Farhang, Salt Lake City, UT (US); Isaac Erickson, Salt Lake City, UT (US); Erin Scull, Salt Lake City, UT (US); Kevin Foley, Salt Lake City, UT (US)

(21) Appl. No.: 17/841,535

(22) Filed: Jun. 15, 2022

Related U.S. Application Data
(60) Provisional application No. 63/210,859, filed on Jun. 15, 2021.
(60) Provisional application No. 63/219,067, filed on Jul. 7, 2021.

(51) Int. Cl. C12N 5/077; C12M 1/42; C12M 1/34; C12M 1/06

(52) U.S. Cl. CPC C12N 5/0655; C12M 35/04; C12M 41/42; C12M 27/02

(57) ABSTRACT

Disclosed herein are compositions, devices, methods, processes, and systems for culturing of large quantities of mammalian cells in suspension culture. Also disclosed are unique and surprising cell populations derived from the disclosed methods, processes, and systems. In many embodiments, the cells are cultured in suspension in liquid culture media that is agitated to maintain the cells in suspension, and agitation increases to maintain cells in suspension while growing and dividing to create cell spheres. The disclosed devices, methods, processes, and systems are useful in tailoring characteristics of the resulting cells based on characteristics of donor subject/initial cells. The disclosed cell populations are useful in treating subjects with cell based therapies in need thereof for various diseases and conditions.

### CROSS-REFERENCE TO RELATED APPLICATIONS

This application claims benefit of priority pursuant to 35 U.S.C. § 119(e) of U.S. provisional patent application Nos. 63/210,859, filed on 15 Jun. 2021 and 63/219,067, filed 7 Jul. 2021, both of which are entitled “NOVEL METHODS FOR PRODUCTION OF THERAPEUTIC MAMMALIAN CELLS AND CELL SPHERES AND COMPOSITIONS OF SAME, and are hereby incorporated by reference in their entireties.

### FIELD

The disclosed compositions, devices, processes, methods, and systems are directed to growth of mammalian cells in suspension in large amounts, and especially for growth of mammalian cells for repair of intervertebral discs.

### BACKGROUND

Disc degeneration is a major cause of low back pain which, in turn, is a driver of health costs/expenditures and patient disability worldwide. Patients who experience disc degeneration have few therapeutic options. Where treatment is available, patients may elect to have surgery to remove the disc in question. However, this type of surgery is both expensive and highly specialized, limiting its availability to patients with sufficient resources and access to the proper facilities and specialists. Moreover, these patients may be forced to wait until their symptoms (and the degeneration) are sufficiently severe to warrant surgery. In such cases, the surgery has mixed outcomes. Even where successful, spinal fusion often leads to accelerated degeneration at adjacent levels. Other therapeutic options, such as traditional approaches including administration of small molecule and biological therapeutics face significant hurdles, such as efficacy and safety in clinical trials. Treatment of disc degeneration by cell therapy may be possible, but such treatments suffer from the problems faced by other cellular therapeutics, such as quickly producing homogeneous cells, at scale, while minimizing cost. Typically, such methods have not been available for production of cell-based therapies.

What is needed are novel therapeutic treatments that effectively and safely address cellular disease and injury, such as degenerative disc disease, while minimizing cost. These treatments need to be manufactured at a sufficiently large scale to support the market size and also reduce overall costs of the product, while maintaining or enhancing efficacy.

### SUMMARY

Disclosed herein are various methods for culturing mammalian cells comprising, collecting a plurality of mammalian cells, introducing the plurality of mammalian cells into a container or vessel, the container or vessel comprising a sphere culture media to create a cell/media mixture; agitating the cell/media mixture at an agitation speed corresponding to a first energy level, wherein the first energy level is sufficient to maintain greater than 90% of the cells in suspension; allowing the plurality of cells to grow, divide, and form cell spheres of a first diameter; increasing the agitation speed to a second energy level, wherein the second energy level is higher than the first energy level and sufficient to maintain the cell spheres in suspension; maintaining the agitation speed to allow the cell spheres to achieve a greater diameter while remaining in suspension. In some embodiments, the mammalian cell may be selected from a progenitor cell, stem cell, or pluripotent cell, and may be derived from muscle, liver, heart, lung, pancreas, bone, thyroid, blood, lymph node, muscle, brain, spinal cord, peripheral nerve, kidney, eye, skin, blood vessel, hair follicle, amnion, chorion, umbilical cord, placenta, cartilage cells, and intervertebral disc cells. In some embodiments, increasing the agitation speed to a second energy level may be linear, nonlinear, or stepwise (for example 2 or more steps) over time. In some embodiments, the sphere culture media may lack a scaffold molecule. In some embodiments, the plurality of cells may be grown in attachment culture prior to introducing the plurality of mammalian cells into a container or vessel comprising the sphere culture media, and/or the cells may attach to the solid surface and allowed to double prior to introducing the plurality of mammalian cells into a container or vessel, the container or vessel comprising a sphere culture media. The mammalian cells may be derived from various mammalian sources and species.

Also disclosed are various methods of culturing a mammalian cell population in dynamic suspension, comprising: introducing the mammalian cell population into a bioreactor containing sphere culture media on a first day to create a cell/media mixture; agitating the cell/media mixture at a first agitation speed; allowing the cell population in the cell/media mixture to form cell spheres; agitating the cell/media mixture at a second speed, wherein the difference between the first speed produces a first shear value that is less than a first max shear value, and the second speed produces a second shear value that is less than a second max shear value.

Disclosed herein are various methods for dynamic culturing of mammalian cell spheres: introducing a mammalian cell population into a bioreactor containing sphere culture media to create a cell/media mixture; agitating the cell/media mixture at a first agitation speed sufficient to prevent or inhibit the cells exiting suspension; allowing the cell population in the cell/media mixture to form cell spheres; maintaining the majority of cells in suspension; isolating and collecting the suspended cell spheres; and thereby dynamically culturing mammalian cell spheres.

Also disclosed are various methods of modifying one or more characteristics of a therapeutic cell population comprising: isolating a population of cells from a donor tissue, wherein the donor has a first attribute with a first attribute score and a second attribute with a second attribute score; determining a desired characteristic of for the therapeutic cell population; selecting a first media parameter based on the first attribute score and/or the second attribute score; selecting a process parameter based on the first attribute score and/or the second attribute score and/or the media parameter; culturing the population of cells in suspension in a container or vessel comprising a sphere culture media; maintaining the population of cells in suspension; allowing the cells to divide and grow to form clonal cell spheres; maintaining the population of cell spheres in suspension; isolating and collecting suspended cell spheres having the pre-determined characteristic; and thereby modifying one or more characteristics of a therapeutic cell population.

Also disclosed are various mammalian cell populations comprising: intervertebral disc cells, wherein greater than 90% of the cells are negative for a surface marker selected from CD24, HLA-DR/DP/DQ, CD45, CD40, CD271, CD80, CD86, or a combination thereof; positive for a surface marker selected from CD44, CD73, CD90, HLA-ABC or a combination thereof.

Also disclosed are mammalian cell populations comprising: intervertebral disc cells, wherein greater than 90% of the cells are express one or more of aggrecan, collagen 1, collagen 2, collagen 6, collagen 14, decorin (DCN), biglycan (BGN), lumican (LUM), and fibromodulin (FMOD); anti-inflammatory effect as seen through activated T-cell assays.

The disclosed cells and methods may include allogenic, autogenic, or xenogenic cells and therapeutic methods.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows growth of cells in various modalities. (Panel A) Cells grown in a static suspension culture modality with methylcellulose exhibit the desired sphere phenotype. (Panel B) Cells grown in waterwheel form into sheets rather than spheres. (Panel C) Cells grown in Erlenmeyer flasks attach to vessel wall rather than in suspension. (Panel D) Cells grown in Wave Bioreactor Bag form large rafts rather than spheres. (Panel E) Cells form spheres when grown at low agitation in stirred tank bioreactors (STRs), however large spheres grow too large in size causing issues with oxygen transport and settling out of solution. (Panel F) A large portion of cells grown at low agitation in STR attach to vessel surfaces impacting growth dynamics. (Panel G) Cells grown at high agitation speeds in STR show some sphere growth but predominantly form single cells. (Panel H) Using a ramped agitation profile with low initial RPM and high final RPM Applicants grow spheres in STR and limit cell attachment

FIG. 2 (Panel A) CFD models show that hydrodynamic conditions scale with different slopes and curvature based on RPM increase in STR. (Panel B) By running STR at various conditions and then modelling aggrecan results using the various hydrodynamic slopes, we are able to see max shear rate (1/s) has the strongest correlation between our investigated hydrodynamic conditions and aggrecan expression (p=0.012).

FIG. 3. (Panel A) The models of max shear and aggrecan (p=0.012), average eddy turbulence dissipation and cell settling (p=0.024), max shear and doublings (p=0.018), and dynamic agitation with sphere size (0.033). (Panel B) Illustration of shifts in hydrodynamic environment as RPM shifts in dynamic culture conditions. By growing cells at various RPM and measuring outputs the effect of hydrodynamic conditions on cells is modelled. (Panel C) Illustration of cell volume fraction calculated from average eddy turbulence dissipation at a single sphere size and RPM. Graphic shows simulated location of cells which can predict cell settling (other RPM, sphere sizes, and scales also simulated). (Panel D) In order to minimize shear forces while also keeping spheres in suspension as they grow in size, CFD is leveraged to develop dynamic agitation profiles.

FIG. 4: Regression analysis of 109 0.25 L STR reactors grown under design-of-experiments conditions. Using these multivariate models, Applicant can ‘maximize desirability’ which tunes the process to results in optimal values for all 11 of our high-risk factors and meets comparability requirements.

FIG. 5 shows studies analyzing donor population process parameters. Panel A: Ranges of acceptable process parameters for donor population. Contour profiles created using variation in two donor attributes and two process parameters are shown with acceptable operating ranges in white and quality attribute failure to shaded by attribute. The four graphs show process parameter 1 and process parameter 2 ranges where donor attributes are fixed to positions: Panel B (low, low), Panel C (low, high), Panel D (high, low), and Panel E (high, high). Together these four graphs inform acceptable ranges for donor attributes and process variability across our investigated donor and process ranges.

FIG. 6 (Panel A): The number of agitation steps impacts process attributes including sphere size, aggrecan expression, and doublings. Panel B: The final RPM used for agitation impacts process attributes including doublings, collagen 1 (via ELISA) and collagen 2 (via PCR).

FIG. 7 (Panel A): Relationship between RPM and various CFD parameters, including max shear. Panel B: Aggrecan ELISA prediction power (R²) by hydrodynamic conditions normalized to max shear. Panel C: Aggrecan ELISA as a function of CFD-calculated max shear in 0.25 L STR.

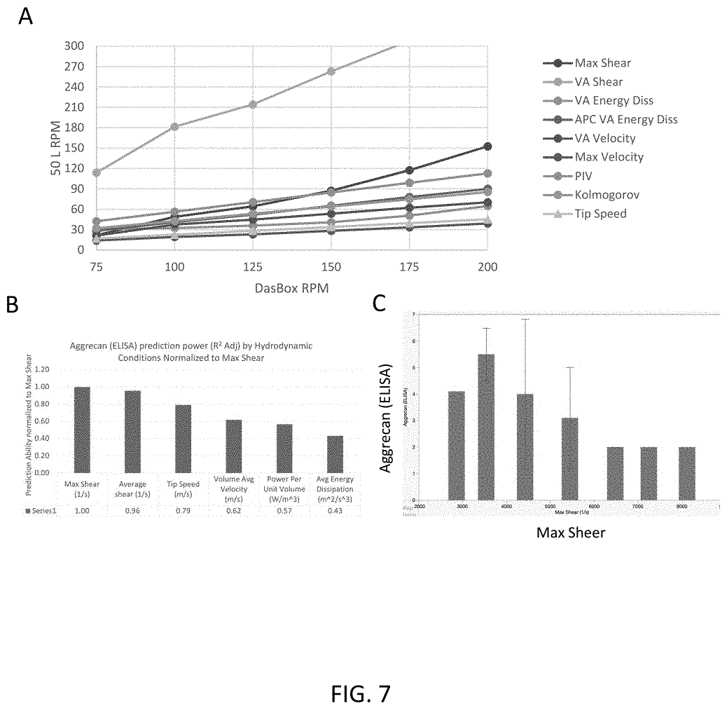

FIG. 8 (Panel A) Diagram of split stream growth where 5 cell lines were grown in both 0.25 L STR and static suspension (“Cellstack”) modalities. (Panel B) Comparable sphere growth in static suspension culture and STR modalities when using ramped agitation. (Panel C) Relative sphere size, doublings, and aggrecan expression from day 2 to end of culture across static suspension culture and 0.25 L STR modalities are comparable. SDEV bars normalized to mean relative value of 0.25 L STR. (Panel D) Identity of cells as measured by flow cytometry is comparable between 0.25 L STR and static modalities

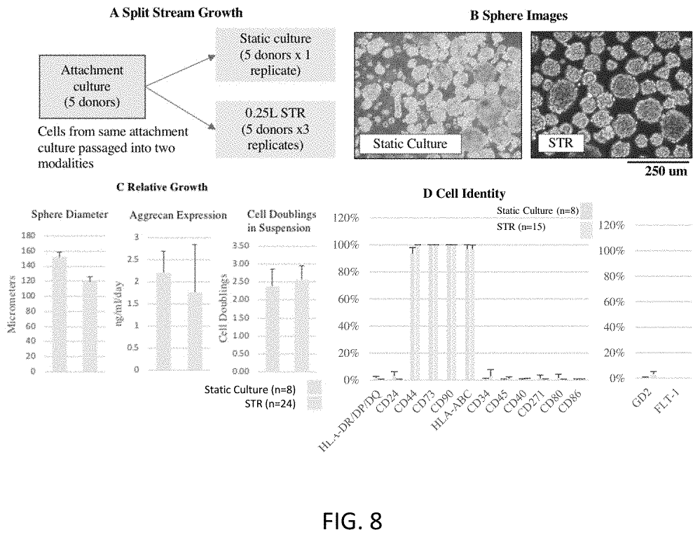

FIG. 9 shows results from studies analyzing the present cells in vivo animal studies. (Panel A) The rabbit study design starts with disc injury, 2 weeks later the discs are dosed, 6 weeks after dosing the study is terminated. (Panel B) Mean percent change in disc height index (DHI) from dosing to termination is shown with standard error. After dosing, the disc height increased slightly in the sham group, more in the vehicle groups, and more substantially in the Cell Therapy groups. ‘*’—indicates statistically significant difference from sham with LSMeans Differences Student's t at a=0.05. (Panel C) Disc Histology shows height restoration and increase in hydration (white areas within center of red disc) for discs injected with Cell Therapy compared to vehicle or sham.

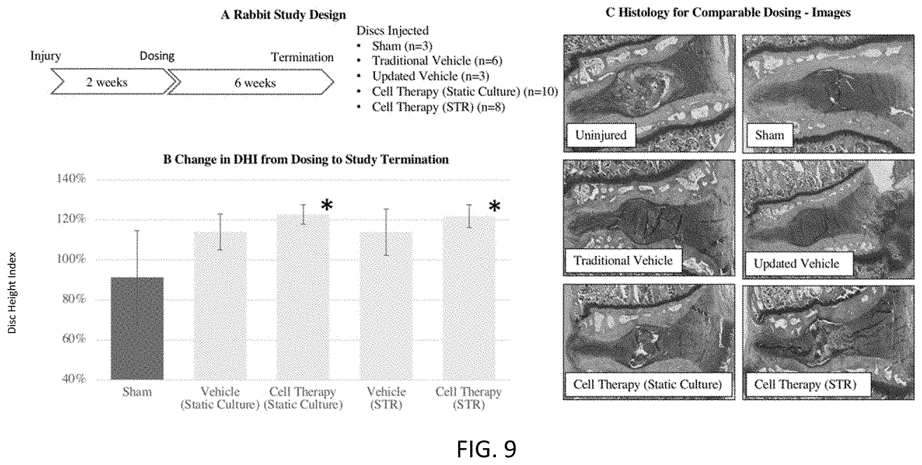

FIG. 10 (Panel A) is a diagram showing split stream growth where cells from a single donor split and grown into two streams—half of the cells grown in small-scale bioreactor (0.25 L) and half of the cells grown in 50 L bioreactors. Panel B shows that comparable sphere growth observed in small-scale and large-scale reactors (Panel C) Relative sphere size and doublings from day 2 to end of culture across 0.25 L and 50 L STR are comparable (Panel D) Identity and purity of cells as measured by Flow cytometry is comparable between 0.25 L STR and 50 L STR.

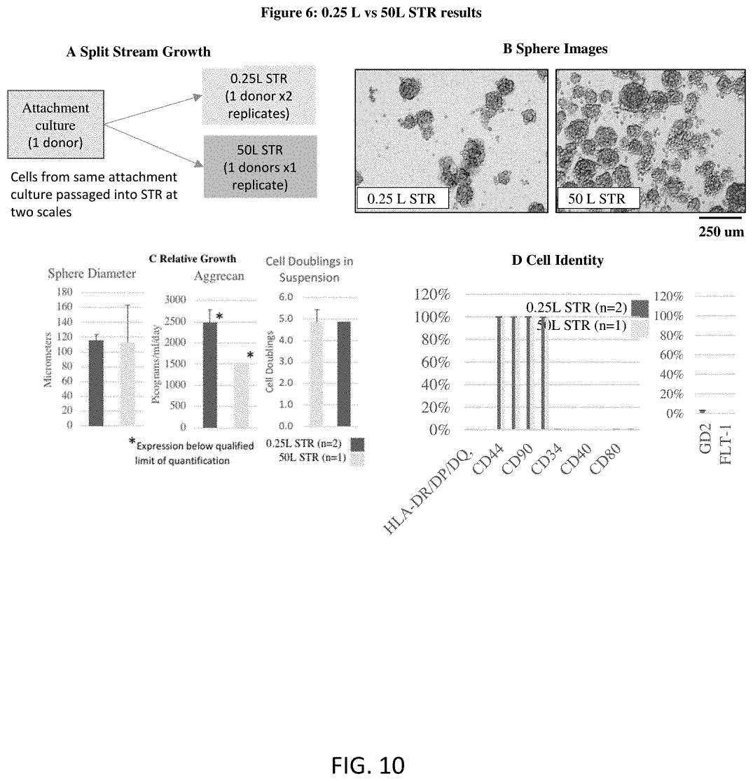

FIG. 11 shows results from flow cytometry analysis of 5 lots of discogenic cells from distinct donors generated either using the static flask method (red) or using STR (blue). Graphs show counts of forward scatter, which is proportional to cell size. Cells generated using STR have a more uniform profile that is smaller in size.

FIG. 12 shows evaluation of ECM production from Discogenic Cells generated using different methods. Analysis included both Collagen I and total collagen, Aggrecan and sGAG (the side chains present in all proteoglycans), as well as a variety of non-traditional ECM molecules that are generated by the cells in culture.

FIG. 13 shows assessment of immunomodulatory properties of discogenic cells. PMBCs are stained with CFSE dye and proliferation is assessed on CD4+ cells via flow cytometry (Panel A); a histogram is generated of un-proliferated and proliferated PMBCs (Panel B). Panel C shows overlay histogram of PMBCs cultured with and without Discogenic Cells indicating inhibition of proliferation by lower CFSE signal with Discogenic Cells. Assay range is also demonstrated by the histogram of unstained and CFSE-stained cells. Relative proliferation of multiple PMBC donors cultured with and without Discogenic Cells.

### DETAILED DESCRIPTION

Existing mammalian cell culture techniques and systems for growth of non-adherent mammalian cells and/or cell spheres suffer from various drawbacks and are inadequate for maintaining the required cell growth environments while supplying sufficient numbers of cells need for treating all subjects in need thereof. For example, existing methods rely on culturing cell spheres using magnetic levitation, scaffolds, and/or viscous carriers. These techniques are incapable of maintaining a standard culture environment for more than a small number of cells produced from a single, static, culture device. In some cases, for example growth in static cultures comprising scaffolds or viscous carriers, results in a fraction of the population developing under attachment culture, due to the fact that it is not possible to completely prevent collecting cells that may have adhered to an interior surface during the culturing process. This results in a more heterogenous population of cells—some of which were grown in adherent culture and some grown as adherent cells.

To obtain sufficient numbers of cells, manufacturers must combine cells grown in separate static culture devices. Static culture and devices for same typically include a scaffold and/or viscous substrate and lack mixing of media and/or cells, in most embodiments, media may be changed periodically in a static culture, but the media is not agitated. As noted above, manufacture of cells by static culture may introduce heterogeneity within the therapeutic dose, and lessen the therapeutic value/effectiveness of that heterogeneous dose. Pooling and isolation of therapeutic cells from static culture also greatly increases the time, effort, and cost of producing mammalian cells for culture, while also reducing the efficacy and potency of the produced cells. Finally, present methods for culturing cell spheres also lack methods for monitoring culture conditions (media, gases, pH, etc.) and refreshing/replacing media components, so that cell densities are often very limited.

Disclosed herein are processes, methods, and systems for large scale growth of therapeutic, mammalian cell populations that allow for rapid growth of large numbers of doses with enhanced homogeneity. The disclosed processes, methods, and systems, because they allow for monitoring conditions and refreshing/replacing various media components, also provide for extended culture times, leading to greater expansion of the therapeutic cell population, growth to higher cell density, less manipulation/handling, and lowered risk of contamination. The disclosed methods, processes, and systems may be used to create distinctive, non-native cell populations from a wide range of tissues and cell types, including stem cells, progenitor cells, and pluripotent cells from brain, liver, kidney, cartilage, muscle, heart, lung, bone, blood, tendon/ligament, pancreas, thyroid, lymph node, spinal cord, peripheral nerve, eye, skin, blood vessel, hair follicle, amnion, chorion, umbilical cord, placenta, cartilage, and intervertebral disc tissue. The disclosed cell populations also possess unique and beneficial characteristics that allow them to be used in research, drug development, and treatment for a variety of diseases, disorders, and conditions.

The disclosed processes, methods, systems, and cell populations may be used to treat or prevent a variety of damage, injury, diseases, disorders, and conditions affecting a variety of cells, tissues, organs, and systems. In many embodiments, the disclosed cells may be useful in treating degenerative disc disease, and may aid in preservation and/or restoration of intervertebral disc height (i.e. the distance between adjacent vertebra), normalization of tissue architecture, etc.

Applicants have developed the present processes, methods, and systems after careful analyses and comparison of various cell culture methods and systems capable of maintaining cells in suspension without scaffold molecules. Applicants discovered that growth, at large scale, of mammalian cell populations, especially cell types whose phenotype is dependent on growth as a cell sphere in the absence of attachment to a solid surface, requires specialized, and changing culture conditions. These changing conditions allow for (1) growth of cell spheres from single cells and (2) maintenance of the cells and cell spheres in suspension, while (3) minimizing disaggregation of cell spheres, (4) minimizing high shear forces that may damage cells and spheres, and (5) settling of even large cell spheres.

Applicant's methods and systems disclosed herein, in one embodiment, include the use of stirred tank bioreactors (STRs) for maintaining the cells in suspension. The disclosed STR culture environment supports creation of dense populations with surprisingly enhanced characteristics relative to other methods—including those that may involve movement of culture media, such as wave rocking bioreactors, shake flasks, and flasks with internal waterwheels. In most cases, bioreactor, may refer to a device or method for growing cells wherein the media is continuously agitated and/or mixed, and may be exchanged, replenished, etc. over the culture period. Moreover, the present methods provide for substantially improved production of mammalian cell spheres relative to existing methods, especially those methods that rely on static culture (i.e. without mixing/movement of culture media) and/or use of scaffolds. In many embodiments, the disclosed methods and systems employ continuous or intermittent fluid movement, while avoiding the use of a scaffold, to maintain and promote suspension of cell spheres. In most embodiments, the disclosed methods and systems comprise the step of growing cell spheres in STR and or an STR device to minimize settling of cell spheres. The disclosed methods and systems maintain size, shape, and quality (e.g. efficacy, homogeneity, etc.) of the disclosed cell spheres. This, in turn, helps maintain and promote homogeneity of many of the beneficial cell quality attributes identified previously in prior, static, scaffold culture methods.

As noted above, Applicants recognized that several aspects of existing cell sphere production methods were unsuitable for scale up and/or production of large amounts of therapeutic cells. Specifically, growth in a scaffold material, while resulting in populations of cells with beneficial characteristics, was costly, labor intensive, prone to contamination, and resulted in heterogeneous cell population. Heterogeneity of cell populations resulted from various circumstances, such as differing microenvironments individual cell spheres were exposed to in the static cultures, differences between populations of cells grown in separate devices and at different times (i.e., macroenvironments), changing culture environments within an individual device, etc. Thus, Applicants investigated whether it was possible to grow cell spheres without a scaffold, while maintaining specific cell characteristics necessary for therapeutic efficacy.

Surprisingly, Applicant show herein that scaffold materials are not required for growth, development, and maintenance of cell spheres and characteristics associated cell spheres. Applicants analyzed various culture methods for the ability to grow and maintain mammalian cell spheres in suspension, such as wave reactors, Erlenmeyer shake flasks, waterwheels, and STR. Unfortunately, the investigated methods were unsuitable for growing the desired population of cell spheres with characteristics matching prior methods, and results proved unreliable. Specifically, large portions of the cells cultured from these methods adhered to solid surfaces within the devices, grew as large masses or sheets of cells (instead of spheres), or remained as single cells.

To aid in developing the presently disclosed methods, processes, and systems, Applicants used Computational Fluid Dynamics (CFD) to investigate, analyze, and compare various culture methods capable of supporting dynamic flow of mammalian cells and culture media. CFD uses fluid mechanics to understand fluid flow in a given system.

After analyzing various dynamic culture conditions and systems as being unsuitable for growth of mammalian cell spheres, applicants elected to investigate modifications to stirred tank reactor conditions for minimization of cell clumping, cell sheeting, and surface attachment. CFD was performed to optimize conditions for a small scale reactor, and also for scale up of reactor size while maintaining desired conditions.

Results from the small scale CFD studies were used to optimize the STR and also scale the system, process, and methods to larger systems and devices, while maintaining desired cell characteristics. Initial small scale studies were performed at about 0.25 L. These conditions were then optimized, using CFD, for maintenance of selected cell characteristics identified in static, scaffold-based methods. These conditions were maintained by adjusting hydrodynamic conditions, using CFD, for much larger cultures, for example 50 L STR cultures.

Applicants' growth of mammalian cells under the presently disclosed hydrodynamic conditions resulted in development, from single cells, of cell spheres, as well as their progressive growth, while maintaining those spheres in suspension as they increase in cell number. In addition, the presently disclosed methods and systems were capable of producing cells with similar and/or enhanced characteristics relative to prior static culture methods. Moreover, the disclosed cell populations possess enhanced homogeneity relative to other methods. In addition, the disclosed methods, processes, and systems provide for continuous monitoring of cell culture data (pH, dissolved oxygen, DO, etc.)—this continuous monitoring also allows for maintaining optical culture conditions throughout the term of growth/culture.

Disclosed herein are novel methods and systems allowing for the expansion and growth of mammalian cells, in large numbers. The disclosed cell culture processes, methods, and systems may include active movement of culture media sufficient to maintain the cells and/or cell spheres in suspension. In many embodiments, the disclosed culture processes, methods, and systems are useful in preventing or reducing settling of cells and/or cell spheres onto a surface, where the cells and/or cell spheres may grow and attach to the cell surface.

The disclosed processes, methods, and systems provide for movement of culture media and cells/cell spheres. In many embodiments, the disclosed methods, processes, and systems may provide for culturing the disclosed therapeutic cells in a bioreactor that may allow for growth of cells and/or cell spheres in culture media that is actively mixing or moving. In many embodiments, movement of the culture media may vary over the culturing period. In many embodiments, the amount of energy used to facilitate movement of the culture media may increase over time, this may be referred to as ramping. In many embodiments, the dynamic movement of culture media may be referred to as culture media agitation and ramped agitation may be referred to as dynamic agitation.

The disclosed methods and systems are useful in maintaining phenotypes, biomarkers, and characteristics of various cells that are can be grown in suspension, for example stem cells, progenitor cells, and the like. In many embodiments, the disclosed methods and systems allow for growth and expansion of cells typically grown as cell spheres or clusters, while maintaining their phenotype and characteristics. In many embodiments, the characteristics of cells and cell populations grown under the disclosed conditions and methods may be significantly enhanced relative to cells grown using other methods and processes, for example static methods and/or non-dynamic culturing.

The disclosed methods and systems are useful in expansion and growth of large-scale, suspension culture of mammalian cells through the movement of culture media, cell spheres, and cells suspended therein. In many embodiments, the disclosed methods and systems provide maintaining mammalian cells in suspension, both single cells, small, multi-cell clusters, and cell spheres. The disclosed methods and systems are useful in allowing single cells to form cell spheres while maintaining the cells and spheres in suspension. In many embodiments, the disclosed mammalian cells possess phenotypes, biomarkers, and characteristics that are similar to or enhanced relative to cells grown in non-dynamic culture, for example cells grown in solid matrix.

The disclosed methods and systems provide for expansion and growth of large numbers of therapeutic cell populations. In many embodiments, the disclosed methods and systems provide for therapeutic mammalian cells that display substantially homogeneous biomarkers and characteristics. In many embodiments, the disclosed therapeutic mammalian cell population displays low heterogeneity in terms of phenotype, biomarker, and characteristic, for example gene expression, extra-cellular matrix production, anti-inflammatory signaling, surface marker display, sphere size, etc.

Disclosed herein are methods and systems for production of large numbers of homogeneous populations of therapeutic mammalian cells. In many embodiments, the disclosed cells are more homogeneous, more effective, and more potent, in vitro and in vivo, than similar cells produced from other methods for example other methods that include scaffold molecules and/or do not include agitation or movement of culture media, as well as methods that include agitation that does not vary over time. The disclosed methods, processes, and systems may avoid or reduce the need for a solid or semisolid scaffold to be included in the culture media.

The reduction or absence of a scaffold may allow for more homogeneous growth of the disclosed cells, and may prevent or reduce the growth of a sub-population of cells that may grow in contact with a solid surface, that is grow in attachment or adherent culture. In many embodiments, the disclosed processes may result in cell populations that are more homogeneous in size, character, identity, etc. In many cases the more homogeneous cell populations may comprise substantially more of one population of cells than another population.

The disclosed methods and systems may result in a substantially homogeneous population of cells possessing higher potency and less variability, for example in terms of one or more characteristics (such as one or more of size, doublings, surface marker expression, etc.), than the other methods. In many embodiments, mammalian cells derived from other methods may be comprised of two or more subpopulations. In many embodiments, cell populations produced from other culture methods, for example culture methods wherein the culture media includes a scaffold, may be heterogeneous and include substantial populations of cells from two or more sub-populations. In some embodiments, a substantial portion of, for one example a cell population, may be greater than about 20%, 30%, 40%, 50%, 60%, 70%, 75%, 80%, 85%, 90%, or 95% of the total.

Disclosed herein are novel compositions of therapeutic mammalian cell populations. In many embodiments, the disclosed therapeutic cell populations possess and express enhanced characteristics, biomarker expression, and other phenotypes. In many embodiments, the disclosed therapeutic cell populations display and express unique and desirable phenotypes that may be unseen, unexpressed, diluted, muted, or masked in cell populations obtained through other culture methods, for example methods that include growth in a scaffold molecule and/or culturing in semisolid, static, and/or un-mixed culture media, and/or mixed/agitated media where agitation does not vary over time.

The disclosed mammalian cells may be various cell types including progenitor cells, stem cells, or pluripotent cells. Stem and progenitor cells of various types are well known in the art, for example as described at Adv Drug Deliv Rev, vol. 60, no. 2, pp. 199-214, Jan. 14, 2008. In many embodiments, the disclosed mammalian cells may be derived from or grown as cell spheres, for example cell spheres grown in suspension culture.

The disclosed cells may be mammalian cells useful in various cell and tissue-based therapies. In many embodiments, the disclosed therapeutic cell populations may be used in allogenic, autogenic, or xenogenic therapies. The disclosed cells, compositions, and methods may be useful in treatment of one or more degenerative diseases.

The disclosed therapeutic mammalian cells may be expanded and modified cells derived from various tissues. In some embodiments, the cells are derived from human nucleus pulposus cells and tissue, and may be used to treat degenerative intervertebral disc disease. In many embodiments, the intervertebral discs may be damaged, diseased, or degenerating, or at risk therefore. In many embodiments, the disclosed cells may be used to treat a subject with a painful intervertebral disc or discs, for example intervertebral discs of the lumbar, thoracic, and cervical regions. In many embodiments, the disclosed cells may be used in the prevention of pain, damage, disease, or degeneration of intervertebral discs, where pain, damage, disease, or degeneration is imminent or anticipated.

One embodiment of the disclosed processes, methods, and systems provide for production of a therapeutic population of mammalian cells derived from intervertebral disc tissue, for example nucleus pulposus cells and annulus fibrosus cells. In many embodiments, the cells may be derived from allogenic human donors. In many embodiments, the disclosed therapeutic cell populations possess one or more modified characteristics that are well-suited treating and preventing various intervertebral disc disorders, conditions, and diseases, for one example degenerative disc disease.

### Cells

The disclosed methods and systems may be useful in enhancing growth, homogeneity, and efficacy of cells from various mammalian tissues. The disclosed cells may be isolated from various mammalian sources. In many embodiments, the disclosed cells are derived from various mammalian tissues. In many embodiments the disclosed mammalian tissues may include one or more of neurological, immunologic, muscle, bone, blood, cartilage, etc. In many embodiments, the cells may be derived from intervertebral disc, annulus pulposus, nucleus pulposus, heart, liver, kidney, lung, pancreas, articular cartilage, bone, thymus, thyroid, blood, brain, spinal cord, peripheral nerve, eye, skin, blood vessel, hair follicle, amnion, chorion, umbilical cord, placenta, cartilage, or lymph node. In some embodiments, the cells are derived from cartilage, tendon, or ligament. In some embodiments, the disclosed cells are derived from intervertebral disc tissue. In these embodiments, the cells may be derived from annulus pulposus and/or nucleus pulposus. In many embodiments, the disclosed cells are derived from nucleus pulposus.

### Donor Attributes

The disclosed therapeutic mammalian cells may be derived from various sources. In one embodiment, the cells are derived from human donors. The donors may exhibit and be scored on various attributes including age, sex, weight, height, body mass index (BMI), health status, etc. In many embodiments, donors may be from about 16-65 years in age, body mass index between about 0-50, weight between about 75-600 lb and height between about 4′5″ to 7′7″. In many embodiments, a donor attribute may include the time between death of the donor and processing of the cells, for example about 0-36 hours. In some embodiments, donor attribute may include health history, for example smoking (packs/day and duration), history of disease for example diabetes, cancer, systemic disease, and other relevant medical information. In some embodiments, the donor attribute may describe health of dissected tissue, for example a disc score capturing the level of fibrosus observed in the dissected tissue, in a score of 1 to 5, with 5 being very fibrous, desiccated, and rough in appearance, and amount of tissue in grams (1-50 grams).

### Culture Vessel for Suspension Growth

The disclosed cells may be grown in suspension in various vessels or containers. In many embodiments the vessel may be configured to hold 1 or more liters of culture media, for example more than 0.1 L, 0.25 L, 1 L, 2 L, 3 L, 4 L, 5 L, 6 L, 7 L, 8 L, 9 L, 10 L, 20 L, 30 L, 40 L, 50 L, 60 L, 70 L, 80 L, 90 L, 100 L, 150 L, 200 L, 300 L, or 400 L and less than about 500 L, 400 L 300 L, 200 L, 150 L, 100 L, 90 L, 70 L, 60 L, 50 L, 40 L, 30 L, 20 L, 10 L, 9 L, 8 L, 7 L, 6 L, 5 L, 4 L, 3 L, 2 L, 1 L, 0.25 L or 0.1 L. In many embodiments, the disclosed vessel or container may be referred to as a bioreactor. In many embodiments, the bioreactor device may include one or more mechanism for inducing or maintaining movement of culture media. In many embodiments, the disclosed bioreactor may include one or more impellers, propellers, or similar devices that may aid in movement of the culture media. The speed at which media is moved/agitated in a bioreactor may be a function of energy used to drive the impellers. In some embodiments, the power may increase over time, for example in one or more steps or as a linear function of time. In many embodiments, the linear function may have one or more plateau where power may not increase, for example at the beginning, end, or middle of the culture period. In some embodiments, steps may be connected by a linear or gradual increase in power from a lower step to a higher step.

The vessel or container may be formed from many substances. In some embodiments, the inner surface of the container or vessel may be coated. In some embodiments, the inner surface may be of a substance or may include a coating that may prevent or reduce adherence of the disclosed mammalian cells to that surface. In some embodiments, the container may contain a flexible vessel, such as a bag, for growth of the disclosed cells. In these embodiments, the bag may include a surface modification to prevent or reduce cell adherence.

The disclosed vessel or container may be configured to aid in mixing of the culture media. In many embodiments, the vessel or container is configured to minimize areas or volumes where movement of the culture media may be reduced sufficiently to allow cells or spheres to settle to the bottom of the container or vessel, or adhere to an inner surface.

### Agitation

While various methods are available for large scale production of mammalian cells using mixing and/or movement of cell culture media, existing methods and conditions are not capable of producing cell spheres and maintaining the cell spheres in suspension.

Applicants herein disclose methods for growth and maintenance of cell spheres in suspension for production of a therapeutic cell population with a substantially homogenous characteristics. Disclosed herein are containers and vessel that may include one or mechanisms useful in moving, agitating, and/or mixing a liquid, for example the disclosed culture media, within the vessel. In many embodiments, the disclosed mechanism is an impeller, propeller, or similar device, wherein the energy of mixing is moderated to (1) support growth and development of cell spheres, (2) prevent cells and cell spheres from precipitating out of suspension, and (3) minimizing or preventing adherence of cells to an interior surface. In many embodiments, the mixing or agitation mechanism may be controlled to allow for changing and/or selecting one or more speeds of movement of the culture media.

Applicants herein disclose methods for growth and maintenance of cell spheres in suspension wherein conditions for growth are optimized using computational fluid dynamics or CFD. In many embodiments, CFD may be used to select growth conditions, including agitation, media, propeller/impeller speed and shape, vessel configuration, vessel volume, etc. to achieve one or more cell characteristics, for example cell and sphere size, biomarker expression, doubling number and time, etc.

The presently disclosed agitation or mixing speed may be varied over time to aid in cell and/or sphere growth. In most embodiments, the agitation speed may increase during culturing and growth of disclosed cells. In many embodiments, selection of agitation speed may be selected based on growth rate, doubling number, doubling rate, rate of change of sphere size, sphere size, biomarker production, and/or expression level of one or more biomarkers.

The disclosed agitation or mixing speed may be increased over time to enhance growth of cell spheres, stability of cell spheres, maintain suspension of cell spheres, and/or minimize settling of cell spheres to a bottom of the vessel and/or adherence of cells or cell spheres to a vessel surface. In most embodiments, the agitation speed may be kept below a maximum shear and above a rate that provides for a Reynolds number of 2500 or more. Reynolds numbers as used herein refer to unit-less value based upon the density of a fluid, its speed of flow, dynamic viscosity and a characteristic linear dimension. Most systems are fully turbulent at a Reynolds number of about 10,000.

The disclosed agitation speed varies over the duration of culturing of the disclosed cells. In many embodiments, the starting agitation speed/energy may be selected based on one or more parameters selected from shear rate, volume average velocity, power per unit volume, energy dissipation. In many embodiments, the starting agitation speed/energy may be selected to maintain single cells in suspension while allowing cell spheres to form and grow. In embodiments where an impeller or propeller is used to mix and/or agitate the culture medium, agitation speed may also be selected based upon impeller tip speed.

Various parameters may be used to aid in selection of an impeller speed. In many embodiments, the parameter is selected from one or more of average energy dissipation (in one example 1×10⁻⁶-1×10⁻⁴ m²/s³), power per unit volume (in one example 0.05-130 W/m³), maximum shear rate (in one example 500-10,000 1/s), average shear rate (1/s), volume average velocity (in one example 0.001-0.2 m/s), maximum velocity (in one example 0.05-1.5 m/s), eddy size (in one example Komolgorov length, 120-20 μm), volume average shear (in one example 0.1-25), volume average energy dissipation (in one example 1×10⁻⁶-1×10⁻²), tip speed (in one example 0.1-2 m/s), and revolutions per minute (RPM from about 10-1000). In some embodiments, the average energy dissipation may be greater than about, 1×10⁻⁷, 1×10⁻⁶, 1×10⁻⁵, 1×10⁻⁴, or 1×10⁻³ m²/s³ and less 1×10⁻², 1×10⁻³, 1×10⁻⁴, 1×10⁻⁵, or 1×10⁻⁶ m²/s³. In some embodiments the power per unit volume may be greater than about 0.01, 0.05, 0.1, 0.5, 1.0, 5.0, 10, 20, 30, 40, 50, 60, 70, 80, 90, 100, 110, or 120 W/m³ and less than about 150, 140, 130, 120, 110, 100, 90, 80, 70, 60, 50, 40, 30, 20, 10, 5, 1.0, 0.5, 0.1, or 0.05 W/m³. In some embodiments, the maximum shear rate is greater than about 500, 1,000, 1500, 2000, 2500, 3000, 3500, 4000, 4500, 5000, 5500, 6000, 6500, 7000, 7500, 8000, or 8500 s⁻¹, and less than about 10,000, 9500, 9000, 8500, 8000, 7500, 7000, 6500, 6000, 5500, 5000, 4500, 4000, 3500, 3000, 2500, 2000, 1500, or 1000 s⁻¹. In some embodiments, the average shear rate of greater than about 10, 50, 100, 150, 200, 250, 300, 350, 400, 450, 500, 600, 700, 800, 900, 1000, 1100, 1200, 1300, 1400, 1500, 2000, 3000, 4000, or 4500 s⁻¹, and less than about 5000, 4500, 4000, 3500, 3000, 2500, 2000, 1500, 1000, 900, 800, 700, 600, 500, 400, 300, 200, 100, or 50 s⁻¹. In some embodiments, the volume average velocity is more than about 0.0001, 0.0005, 0.001, 0.005, 0.01, 0.05, 0.06, 0.07, 0.08, 0.09, 0.1, or 0.15 m/s and less than about 0.2, 0.15, 0.1, 0.09, 0.08, 0.07, 0.06, 0.05, 0.04, 0.03, 0.02, 0.01, 0.005, 0.001, or 0.0005 m/s. In some embodiments, the maximum velocity is more than about 0.01, 0.02, 0.03, 0.04, 0.05, 0.06, 0.07, 0.08, 0.09, 0.1, 0.2, 0.3, 0.4, 0.5, 0.6, 0.7, 0.8 or 0.9 m/s and less than about 1.0, 0.9, 0.8, 0.7, 0.6, 0.5, 0.4, 0.3, 0.2, 0.1, 0.09, 0.08, 0.07, 0.06, 0.05, 0.04, 0.03, and 0.002 m/s. In some embodiments, the eddy size is greater than about 10, 15, 20, 25, 30, 35, 40, 45, 50, 55, 60, 65, 70, 75, 80, 85, 90, 95, 100 or 110 μm and less than about 150, 130, 120, 110, 100, 95, 90, 85, 75, 70, 65, 60, 55, 50, 45, 40, 35, 30, or 25 μm. In some embodiments, volume average shear is greater than about 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.1, 2.2, 2.3, 2.4, 2.5, 2.6, 2.8, 2.7, or 2.9 and less than about 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.1, 0.09, 0.08, 0.07, 0.06, 0.05. In some embodiments, the volume average energy dissipation is greater than about 1×E-6, 1×E-5, 1×E-4, 1×E-3, or 1×E-2 and less than about 1×E-1, 1×E-2, 1×E-3, 1×E-4, or 1×E-5. In some embodiments, the tip speed is greater than about, 0.05, 0.06, 0.07, 0.08, 0.09, 0.1, 0.11, 0.12, 0.13, 0.14, 0.15, 0.16, 0.18, 0.17, 0.19, 0.20, 0.21, 0.22, 0.23, 0.24, or 0.25 m/s and less than about 0.3, 0.25, 0.24, 0.23, 0.22, 0.21, 0.20, 0.19, 0.18, 0.17, 0.16, 0.15, 0.14, 0.13, 0.12, 0.11, 0.10, 0.09, 0.08, 0.07, or 0.06 m/s. In some embodiments, the starting or ending RPM of the impeller(s) is greater than about 10, 20, 30, 40, 50, 60, 70, 80, 90, 100, 110, 120, 130, 140, 150, 160, 170, 180, 190, 200, 250, 300, 350, 400, 450, 500, 600, 700, 800, 900, 1000 RPM, and less than about 1250, 1000, 900, 800, 700, 600, 500, 400, 300, 350, 250, 200, 190, 180, 170, 160, 150, 140, 130, 120, 110, 100, 90, 80, 70, 60, 50, 40, 30, or 20 RPM.

As described elsewhere, various parameters may be increased/changed during the suspension culture period to maintain to optimize and/or tune performance characteristics/attributes of the resulting cell population. In many embodiments, the amount of energy applied to mix the culture (e.g., impeller or mixer speed) may increase over the course of the culture period. In many embodiments, the impeller speed may be less increased over time, which may also result in an increase of shear, avg. shear, and/or max shear over time. In one embodiment, the max shear may initially be about 1000-3000 s⁻¹ and increase over time, but have an ending value of between about 4000 and 8000 s⁻¹. In one example the initial max shear may be about 2863 s⁻¹ and may be increased to about 4400 s⁻¹ at day 4 of suspension culture.

The disclosed agitation speed may increase during the culture period or culturing period. The culture period may refer to the time between inoculation of a culture media with a number of mammalian cells (single cells and/or cell spheres) and the time when cell spheres are collected. In many embodiments, the culture media may be inoculated with single cells or small cell spheres (between 2 and 50 cells). In many embodiments, the culture period may end with collection of the cells, that may be predominantly cell spheres of a size between about 50-300 microns. In most embodiments, the increase in agitation speed may be steady, and substantially linear, or the increase may be step-wise, for example including 2, 3, 4, 5, 6, 7, 8, 9, 10, or more steps between inoculation and collection. In many embodiments, the disclosed method may include three different steps of agitation speed over the culture period; that is an initial setting when the culture is inoculated, a second setting, a third setting, followed by collection of the cell spheres. In one embodiment, for example where the culture vessel is 200-300 ml, the disclosed methods may include starting and ending RPM for a 250 mL vessel of 75-275 RPM.

The initial agitation speed may be selected to provide for both maintenance of the cells in suspension and allowing formation of cell spheres—that is, avoidance of forces sufficient to separate or tear apart cell spheres.

### Media

### Other Process Parameters

The disclosed cells may be grown in suspension culture and various growth parameters adjusted. In some embodiments the seeding density may vary from about 100 to about 1×10⁶ cells/mL. Vessel and growth conditions may also be varied, for example culture time, temperature, volume, dissolved oxygen, pH, media exchange rate, perfusion supplement exchange rate, etc. In various embodiments, the culture time may vary from about 7 to about 25 days. In some embodiments, the final cell density may be about 100 to about 10e6 cells/mL. In various embodiments, the culture temperature may vary from about 35 to about 38° C.). The volume of media in the vessel may also vary from about 50 to about 100%, dissolved oxygen percentage may vary from about 50 to about 110%, pH of the media may vary between about 7.2 to about 7.8. In various embodiments, the media and perfusion exchange rate may be varied from about 0 to about 500%/day.

### Cell Growth/Doublings

The disclosed methods, processes, and systems are useful in enhancing the growth rate and/or expansion capacity of mammalian cells grown in-vitro without loss of potency. In many embodiments, the disclosed methods, processes and systems allow for more cell doublings without manual intervention (i.e. ‘splitting’ cells from one culture vessel into two culture vessels, dissociating cell spheres into single cells to regrow more spheres, etc.). In many embodiments, the disclosed methods, processes, and systems allow for faster growth of more uniform cells and more homogenous cell populations.

The disclosed cells may be grown in attachment culture before or after growth in suspension. In many embodiments, the disclosed cells may undergo two or more divisions before being placed in suspension. In many embodiments, the disclosed population of cells may undergo 2, 3, 4, 5, 6, 7, 8, 9, 10, 11, 12, 13, 14, 15 or more ‘doublings’ before being transferred to suspension culture, where a ‘doubling’ refers to a population of cells increasing by 2-fold or 2×.

When grown in suspension, the disclosed cell populations may double from 1 to 15 times. In many embodiments, the disclosed cells may undergo two or more doublings during growth in suspension, for example more than about 2, 3, 4, 5, 6, 7, 8, 9, 10, 11, 12, 13, 14, or 15 ‘doublings’ before being harvested.

As used herein, a cell population may be described as undergoing one or more doublings, wherein the number of cells in a population ‘doubles’—such that a population that has undergone two ‘doublings’ has increased in number from initial inoculation of the culture by 4-fold or 4×, where x is the starting number of cells.

### Sphere Size

The disclosed methods, processes, and systems provide for growth of mammalian cell spheres. In many embodiments, the disclosed cell spheres are of a size between about 20 μm and 200 μm. In many embodiments, the mean average size of cell sphere produced by the disclosed method is about 50 μm to about 75 μm. In many embodiments, about 80% or more of the disclosed cell spheres are between about 93 μm and 164 μm. In many embodiments, a cell sphere may comprise a plurality of cells derived from a progenitor cell, for example a clonal population. In many embodiments, the cell sphere may comprise more than about 50, 60, 70, 80, 90, 100, 110, 120, 130, 140, 150, 160, 170, 180, 190, 200, 250, 300, 350, 4000, 450, or 500 cells and/or less than about 2000, 1500, 1000, 500, 450, 400, 350, 300, 250, 200, 150, 100, or 50 cells.

### Bio Markers

Applicant's disclosed methods and systems may induce and/or support various gene and/or protein expression profiles. In many embodiments, the gene and protein expression profiles of the disclosed cells may aid in supporting tissue repair or health. In some embodiments, the expressed genes and proteins may aid in repair and/or maintenance of intervertebral discs, for example intervertebral discs at risk for or displaying degenerative disc disease. In many embodiments, this difference in gene expression may be different than that produced by other culture methods, for example culture methods that include one or more viscous scaffolds to maintain the cells in suspension and culture methods that may use one or more techniques for media agitation such as a water wheel or movement of the culture or vessel device. In some embodiments, expression is enhanced for genes and proteins that have been shown to be suppressed in degenerative disc disease.

Biomarkers, including genes, proteins, and compounds, may display enhanced expression as a result of the present methods. The disclosed biomarkers may include one or more related to proteoglycans or collagen. In many embodiments, the disclosed biomarkers may be for one or more small leucine rich proteoglycans. The disclosed gene, protein, or compounds may be selected from one or more of nucleus pulposus markers, extracellular matrix molecules, glycosaminoglycans (GAGs), small leucine rich proteoglycans (SLRPS), aggrecan (NP_001126, XP_001131727, XP_001131734; NP_037359; NP_001356197, XP_006720482; XP_011519615; XP_011519616), collagen 1, 2, 6, 12, and others. In many embodiments SLRPS are glycoproteins that include decorin (DCN; NP_001911; NP_598010; NP_598011; NP_598012; NP_598013; NP_598014), biglycan (BGN; NP_001702), lumican (LUM; NP_002336), and fibromodulin (FMOD; NM_002023.5).

### Expression

Biomarker expression may be measured by various methods. In many embodiments, biomarker expression is measured by one or more of flow cytometry, PCR, RT-PCR, protein assay, glycan assay, ELISA assay, colorimetric assay, etc. In one embodiment, flow cytometry may be performed with fluorochrome-conjugated mouse antihuman monoclonal antibodies. In many embodiments, flow cytometry may include one or more isotype controls. In many embodiments, the disclosed cells may be incubated with one or more antibodies that recognize a surface receptor, marker, or protein. In many embodiments, incubation may be performed at 4° C. for about 20 to 90 minutes in the presence of serum albumin and a human Fc block. Various surface markers may be assayed, such as one or more of HLA-DR/DP/DQ, CD24, CD44, CD73, CD90, HLA-ABC, CD34, CD45, CD40, CD271, CD80, Gd2, Flt-1 and CD86. In some embodiments, dead or metabolically inactive cells may be identified and later excluded from analysis, for example with a compound that helps to differentiate these cells from live cells—for example the compound 7-AAD. In many embodiments, biomarker expression, such as surface marker expression may be quantitated by flow cytometry, and data regarding expression over a population, and less than 100%, 95%, 90%, 85%, 80%, 75%, 70%, 65%, 60%, 55%, 50%, 45%, 40%, 35%, 30%, 25%, 20%, 15%, 10%, 5%, 4%, 3%, 2%, or 1% and more than about 0.1%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, or 95% of the population expresses the bio marker.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, or 0.05% of a given value or range. Whenever the term “about” or “approximately” precedes the first numerical value in a series of two or more numerical values, it is understood that the term “about” or “approximately” applies to each one of the numerical values in that series. In many embodiments, ‘substantially’ or ‘substantial’ may refer to a majority of a portion, for example greater than about 50%, 60%, 70%, 75%, 80%, 85%, 90%, 91%, 92%, 93%, 94%, 95%, 96%, 97%, 98%, 98%, or 99% of the set or population. In some cases, substantial may be used in reference to improvement, in these cases, the improvement may be 10%, 20%, 30%, 40%, 50%, 60%, 70%, 80%, 90%, 100%, 200%, 300% or more.

### Examples

### Investigation of Scaffold-Free Culture Methods

Intervertebral disc tissue obtained from recently deceased mammalian donor subjects. Individual nucleus pulposus cells were isolated from the disc tissue via enzymatic digestion. Single cells were then expanded in adherent culture. Next, cells were split into separate groups, and each group of cells was grown in one type of non-adherence, or attachment independent, culture conditions. Specifically, one group of cells was grown by the previous method containing viscous scaffold in flasks. Other groups were grown in cultures based on: rocking non-stick bags, Erlenmeyer shake flasks, vessels containing internal rotating wheels, and STRs. Each group of cells was seeded into the same culture media cocktail used in previous flask system, but, except for the control flask group, the media lacked methylcellulose.

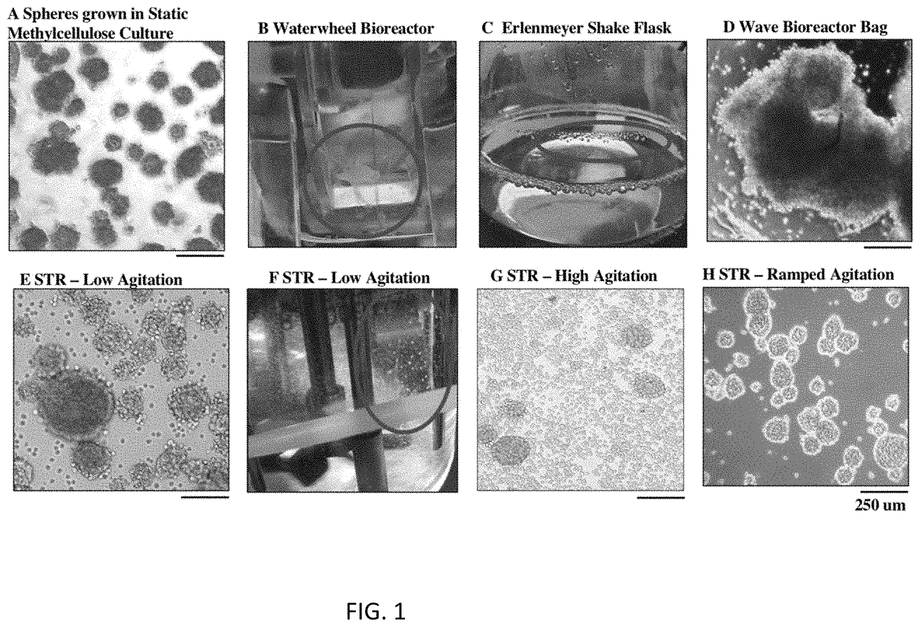

FIG. 1 shows images of the various flasks/vessels/containers/bioreactors and systems. Additionally, FIG. 1 shows microscopic images of one embodiment of the disclosed cells/spheres obtained from the present methods. These micrographs are produced at various magnifications immediately prior to harvest.

At harvest, cell spheres are dissociated into single cells and counted to obtain a final total cell number (using a K2 automated cell counter; Nexcellom). The number of cell doublings was calculated (doublings=3.32 (log(harvest count)−log(inoculation count)) for each modality and recorded. Recorded doubling values were compared to cell doubling in the flask system.

After initial analysis, Applicants identified methods that supported cell growth and sphere formation, without significant cell attachment to vessel surfaces. Cells from these methods were then further processed (washed, concentrated, and frozen at −196° C.) for additional testing.

Cells were grown in four modalities with passive control and one modality with active control. The first modality, and control condition, was static suspension culture in CellSTACKs with an ultra-low attachment coating (Corning) (n=8). In this static culture, media included 0.75% methylcellulose (Benecel A4M, Ashland) to provide a scaffold, prevent cell adhesion and promote sphere growth. In the remaining modalities, cells were suspended with fluid movement in methylcellulose-free media. The second modality was 500 ml non-baffled, glass Erlenmeyer flasks with vented caps (Chemglass, n=2 at 100 and 130 revolutions per minute (RPM)). Flasks were siliconized (Sigmacote siliconizing reagent, Sigma-Aldrich) prior to use in order to mitigate cell adhesion to vessel walls. The third modality was a vented cap waterwheel (PBS 0.1MAG, PBS Biotech, n=2). The fourth modality was a Wave bioreactor (Xuri Cell Expansion System, Cytiva, n=2). The reactor was rocked at a 4-degree rocking angle at 4 full oscillations per min. For each passively controlled modality, temperature was maintained at 37° C. Dissolved oxygen and pH were maintained through a vented cap with 95% air and 5% CO₂ using an incubator.

The fifth modality utilized active control, cells were grown in 0.25 L glass stirred tank bioreactor (0.25 L DASbox Mini Reactor Systems, Eppendorf). STR vessels were siliconized with Sigmacote prior to use in order to mitigate cell adhesion to vessel walls. Three agitation speeds (low, medium, high) were utilized (n=2 per condition) using an 8-blade impeller. Dissolved oxygen and pH were actively controlled using a mixture of CO2, air, 02, and N2 gas overlay.

In each of the 5 modalities, cells were grown for comparable durations. Macroscopic images of the flasks/bioreactors and microscopic images of the cells/spheres were obtained at various magnifications immediately prior to harvest.

TABLE 1
Investigated Culture Modalities

| | | | Working | pH & Dissolved |
|---|---|---|---|---|
| Vessel | Supplier | Suspension Method | Volume (mL) | Oxygen control |
| CellSTACK (Control) | Corning | Methylcellulose Static | 165 | Passive |
| Xuri Cell Expansion System | Cytiva | Rocking Platform | 495 | Passive |
| Erlenmeyer Flask | Chemglass | Orbital Shaking | 125 | Passive |
| Waterwheel | PBS Biotech | Horizontal Axis Wheel | 100 | Passive |
| DASbox (STR) | Eppendorf | Vertical Axis Impeller | 250 | Active |

### Agitation Modeling Study

The 0.25 L STR were run at 22 different conditions, which included 6 with various static agitation profiles, and 16 with ramped agitation profiles. Cell doubling, sphere size, and extracellular aggrecan matrix production was measured for each condition. Then CFD was used to generate hydrodynamic models of fluid forces in the 0.25 L STR (described below). These hydrodynamic models were compared to cell quality outputs using standard least squares regression. Finally, the STR parameters were modified to a new optimal set-point identified using the regression models of hydrodynamic forces and cell quality.

### Cell Doubling

At harvest, cells were dissociated out of spheres and a final cell number was obtained using a K2 automated cell counter (Nexcelom) to assess total growth kinetics. The cell doublings (doublings=3.32 (log(harvest count)−log(inoculation count)) was reported as multiples compared to the doublings found in the static suspension culture.

### Sphere Size

Sphere size was measured via image analysis in ImageJ software [22]. Three photos of concentrated sphere harvest were taken at 40× magnification per vessel and analyzed using a custom ImageJ plugin generated in-house. For each photo, the custom plugin created an 8-bit grayscale image, removed outliers, filled holes, and used watershed to identify spheres and split up overlaid spheres. Finally, the plugin set scale and measurements to capture sphere characteristic data and adjusted for image magnification before the “Analyze Particles” function captured these measurements for all spheres between 1000-7500 pixels in size and 0.15-1.0 in circularity.

### Extracellular Matrix Protein In Vitro Culture Assay

Discogenic Cells were seeded in 96-well round bottom ultra-low attachment plates at 2.5×10⁵ cells/well in DMEM/F12 with 0.5% fetal bovine serum and 50 ug/mL gentamicin. Cell cultures were incubated at 37° C. with 5% CO₂ for 72 hours. Supernatant was removed from the culture for analysis by ELISA assays to determine the concentration of aggrecan (Aggrecan (PG) Human ELISA Kit, Thermo Fisher Scientific). An internal reference control cell line was run with each assay to in parallel verify the assay performance.

### Computation Fluid Dynamic Modeling

Next, detailed models of two STR systems (0.25 L DASbox by Eppendorf and HyPerforma 5:1 50 L S.U.B. by Thermo Fisher Scientific) were created using DesignModeler, Meshing, Fluent & CFD Post (ANSYS 18.1, Ansys) The system was modeled using 7 agitation speeds which aim to capture the impeller's RPM and hydrodynamic conditions scale. Average energy dissipation (m²/s³), power per unit volume (W/m³), maximum shear rate (1/s), average shear rate (1/s), volume average velocity (m/s) and tip speed (m/s) were calculated using a set of assumptions and equations found in the Supplemental Materials.

Cell doublings, sphere size, and aggrecan outputs from the twenty-two STR vessels were modeled using these CFD-derived hydrodynamic conditions and compared to the empirical findings using a standard least squares regression. CFD was also used to calculate the correlation between average eddy turbulence dissipation and distribution of the cells within each part of the reactor (cell volume fraction) assuming the spheres were the maximum observed sphere size from the STR runs.

### Comparison of Static Suspension and STR Modalities

The STR parameters were updated to optimize doublings, sphere size, and aggrecan production values based on the agitation and CFD regression models. The static suspension and new STR culture processes were compared with cells derived from 5 distinct human donors cultured in parallel. A ramped agitation profile was used for the STR, where the agitation rate increased over time to account for the increase in sphere size. One replicate per static suspension condition and 3 replicates per 0.25 L STR were performed. The cells generated from each were compared for key attributes including cell doublings, sphere size, aggrecan expression, and flow cytometry identity. The bioactivity of the cells was also compared in an in vivo rabbit model of disc degeneration.

### Updated Extracellular Matrix Protein In Vitro Culture Assay

For this comparison of static suspension and 0.25 L STR studies, the assay was slightly modified to improve sensitivity. Discogenic Cells were seeded in 96-well v-bottom polypropylene plates at 2.5×10⁵ cells/well in DMEM high glucose with pyruvate supplemented with 1×ITS+premix, 0.35 mM L-proline, 0.17 mM 2-phospho-L-ascorbic acid and 50 ug/mL gentamicin. Cell cultures were incubated at 37° C. with 5% CO₂ for 5 days. Supernatant was removed from the culture for analysis by ELISA assays to determine the concentration of Aggrecan (Aggrecan (PG) Human ELISA Kit, Thermo Fisher Scientific) and Collagen I (Abcam). An internal reference control was run with each assay to verify the assay performance.

### Identity by Flow Cytometry

The cell identity was measured using flow cytometry with fluorochrome-conjugated mouse antihuman monoclonal antibodies, including appropriate isotype controls. The cells were incubated with antibodies at 4° C. for 30 to 60 minutes, in PBS with 0.5% human serum albumin, human Fc block and the following antibodies HLA-DR/DP/DQ, CD24, CD44, CD73, CD90, HLA-ABC, CD34, CD45, CD40, CD271, CD80, and CD86 (BD Biosciences, San Jose, Calif., USA). Forward scatter histograms were generated. Positive expression was assessed in the live cell population using 7-AAD (BD Biosciences) staining to exclude dead cells. The flow cytometry measurements were performed on a CytoFLEX Flow Cytometer (Beckman Coulter Life Sciences, Indianapolis, Ind. USA) and analyzed with FlowJo Software (BD Bioscience). No less than 10,000 events were collected for each analysis.

### Process Tunability Modeling

Five donors were selected to represent our donor population, four donors (Donors 1-4) with donor quality attributes at the edges of our acceptable ranges and one donor (Donor 5) with donor quality attributes at the center of the range. A data table of donor attributes was created to allow for covariate data within the DOE dialog (Table 2), where lower end of the range is represented as −1, the higher end of the range is selected as +1, and the range center point is represented as 0. By creating this covariate table the disclosed data analysis software (as one example; JMP from SAS) automatically creates a Whole Plot with a Random Block Role for each donor in the DOE, in addition to the selected model roles.

TABLE 2
Covariate Donor Attributes

| Donor ID | Donor Attribute 1 | Donor Attribute 2 |
|---|---|---|
| Donor 1 | −1 | 1 |
| Donor 2 | −1 | −1 |
| Donor 3 | 1 | 1 |
| Donor 4 | 1 | −1 |
| Donor 5 | 0 | 0 |

We created a D-optimal DOE (design of experiments) using 2 covariate donor quality parameters, 4 high risk media parameters, and 5 high risk process parameters (Table 3). The media and process setpoints were given a continuous role in the DOE dialog. The DOE was designed with 6 runs of 14 STR conditions in a blocking role. Primary effects, interaction effects, and curvature estimibility were each set to “Necessary” in the DOE dialog. The D-Optimal DOE was created and executed. Outside of the DOE dialog a seventh run was conducted where each of the five cell lines were run in triplicate under center point conditions for media and process parameters. In cases where experimental conditions were not maintained at DOE setpoints an additional STR was run in an eighth run. The seventh and eighth runs were each assigned their own blocking level.

TABLE 3
DOE Input Values

| Input Name | Role | Changes | Coded Values | | | | | |
|---|---|---|---|---|---|---|---|---|
| Donor Attribute 1 | Covariate | Hard | −1 | | | +1 | | |
| Donor Attribute 2 | Covariate | Hard | −1 | | | +1 | | |
| Media Parameter 1 | Continuous | Easy | −1 | | | +1 | | |
| Media Parameter 2 | Continuous | Easy | −1 | | | +1 | | |
| Media Parameter 3 | Continuous | Easy | −1 | | | +1 | | |
| Media Parameter 4 | Continuous | Easy | −1 | | | +1 | | |
| Process Parameter 1 | Continuous | Easy | −1 | | | +1 | | |
| Process Parameter 2 | Continuous | Easy | −1 | | | +1 | | |
| Process Parameter 3 | Continuous | Easy | −1 | | | +1 | | |
| Process Parameter 4 | Continuous | Easy | −1 | | | +1 | | |
| Process Parameter 5 | Continuous | Easy | −1 | | | +1 | | |
| Bioreactor Run | Blocking | Easy | L1 | L2 | L3 | L4 | L5 | L6 |

Data from the eight runs were analyzed using regression and ANN techniques (outlined below). Using either method, optimal conditions were identified for all 11 donor, media, and process setpoints. To test these setpoints, STR were run in triplicate at our process center point and optimal conditions as identified by the ANN and regression models.

The DOE was first evaluated for normal distribution using an Anderson-Darling goodness of fit test. Data that was normally distributed was then analyzed by standard least squares regression with the reduced maximum likelihood method. All primary, interaction, and quadratic effects were included in the initial model. Inputs which did not contribute significantly to the model (p>0.05), or which did not have a higher order effect still included in the model, were removed from the model one at a time in order of their descending p-values. Once only significant effects remained, R-square adjusted and analysis of variance were used to assay model quality. To find optimal process setpoints cell doublings were maximized using a prediction profiler, such as a prediction profiler within the analysis software. Profilers may aid in visualizing response surfaces upon changing one or two input parameters/factors. In many cases, a profile may be a cross-sectional view of the data allowing exploration of various spaces—for example opportunity spaces. Potential multivariate ranges were assessed using a contour profiler available with JMP software.

Results from the tenability DOE demonstrate that donor cells with varied attributes, may be produced with specific performance characteristics, by tuning/selecting process and media parameters. In many embodiments, performance attributes may be selected based on various criteria or the intended use of the cells.

### In Vivo Evaluation of Bioactivity

For in vivo evaluation of bioactivity, female New Zealand White Rabbits (3-4 kg) were used under approval by a private Institutional Animal Care and Use Committee (IACUC). Fourteen rabbits were anesthetized and the lumbar region of the spine surgically exposed. Three lumbar discs (L3-L4, L4-L5, L5-L6) were injured via insertion of an 18-gauge needle 5 mm into the disc. L2-L3 was left undisturbed as a healthy control. Afterwards, muscle and skin were closed using sutures and the animals monitored during recovery. Rabbits were again prepared for surgery two weeks later, and the discs injected with 25 ml of 0 or 67,000 cells in vehicle from two different donors (n=3-6/condition across 2 animals per condition) through a 27-gauge needle. A sham procedure was also performed for comparison (n=3). Half of the cells were generated in static culture containing methylcellulose and mixed with the traditional vehicle (1% sodium hyaluronate with Profreeze (Lonza Bioscience), dimethyl sulfoxide, saline and human serum albumin). The other half of the cells were generated in STR and mixed with an updated vehicle that excludes Profreeze.

X-ray images were obtained of the lumbar spine every 2 weeks. Measurements between 18 boney landmarks were taken in a blinded manner to calculate disc height index (DHI) for the various conditions. DHI and measurement of same is well known in the art as disclosed at L. I. Silverman et al., “In vitro and in vivo evaluation of discogenic cells, an investigational cell therapy for disc degeneration,” Spine J, vol. 20, no. 1, pp. 138-149, January 2020. Also, animal body weight and behavior were noted for any abnormalities. Six weeks after dosing, the animals were sacrificed and the discs explanted, fixed, decalcified, sectioned through the center of the discs and stained with hematoxylin and eosin (H&E), Safranin 0, and Picrosirius Red/Alcian Blue mixture. The slides were evaluated by a board-certified veterinary pathologist for the presence of abnormal tissue or inflammation as well as any potential normalization of tissue architecture.

Scale-Up into Large-Scale STR

A CFD model of the HyPerforma 5:1 50 L S.U.B. (Thermo Fisher Scientific) was generated. RPM was scaled by maintaining maximum shear rate (s−1) between scales. Cells from a single donor were split and in the 0.25 L STR (DASbox, n=2) and a 50 L pilot-scale commercial system (HyPerforma 5:1 50 L S.U.B. by Thermo Fisher Scientific, n=1). The resulting Discogenic Cells were compared for doublings, sphere size, and identity via flow cytometry using methods describes above.

### ECM Production

Serum free ECM culture media was prepared consisting of High Glucose DMEM, 1×ITS+premix (corning), 0.35 mM proline, 0.17 mM ascorbate-2-phosphate, and 25-50 μg/mL gentamicin sulfate. Liquid nitrogen samples were thawed and transferred to a centrifuge tube with media. Samples were centrifuged at 200-400×g for 5 minutes to pellet the cells. The supernatant was aspirated and cell pellet resuspend in 1-3 mL of ECM culture media. Cell concentration was measured using a K2 or cellaca cellometer (Nexcelom) and the concentration of cells adjusted to between 0.5-3*10\hat{ }6 viable cells/mL accordingly. 200-300 μL of each cell suspension was added to each well of an ultra-low attachment 96-well round-bottom plate. Plates, with cells, were incubated in a cell culture incubator (37° C., 5% CO₂). After 3-5 days, cell plates were removed and centrifuged at 200-400×g for five minutes. Supernatant was carefully transferred, without disturbing the cell pellet, to a new 96-well v-bottom plate.

Collagen I Elisa assays were performed according to standard practices. Aggrecan ELISA assays were performed according to standard practices, except that 60-100 μl of sample rather than 50 μl of sample was used, and the volume of incubation buffer adjusted according to be equivalent to the amount of sample volume input (60-100 μl). The total amount of Collagen I and Aggrecan in the cell supernatant was determined using a standard curve prepared for each analyte.

### PCR Assay

Samples were taken either from the cell pellets following in vitro culture within the ECM potency assay or from cells taken from suspension culture harvest prior to dissociation (note: samples can also be taken after dissociation). Fresh cell samples (i.e. taken from cell pellets following in vitro culture) may express different biomarkers at different levels compared to cells taken from suspension culture. In many embodiments, the cell samples may be processed similarly for PCR analysis. Samples were lysed in TRIzol reagent and stored at −80 C prior to analysis. RNA was extracted using a PureLink RNA Microscale Kit. Gene expression was measured using commercial TaqMan assays for ACAN (Hs00153936_m1), COL1A2 (Hs00164099_m1) and COL2A1 (Hs01060325_g1) and normalized to expression of the housekeeping gene HPRT1 (Hs02800695_m1). The reverse transcription to cDNA and the PCR amplification steps were performed in the QuantStudio 5 Real-Time PCR System with a single experiment using 1-step Fast Virus Master Mix (ThermoFisher catalogue 4444436) using the recommended cycling conditions.

### Pellet Weight Assay

Prepare serum free pellet culture media consisting of High Glucose DMEM, 1×ITS+premix (corning), 0.35 mM proline, 0.17 mM ascorbate-2-phosphate, and 25-50 ug/mL gentamicin sulfate. Cells are thawed and resuspended in 1-9 mL of pellet culture media. Cells are counted on a K2 or cellaca cellometer. 3-9 million cells are transferred to a 15 mL tube and cell concentration adjusted to be 1-2 million cells/mL. Cells are centrifuged at 200-400×g for 5 minutes to pellet cells. Pellets are cultured for 2 weeks in incubator at 37 C and 5% Oxygen, and media is changed every 2-3 days. At the end of culturing, cells are harvested and weighed to measure the wet weight of the pellet.

Biochemical Assay to Measure sGAG and Hydroxyproline

Prepare papain digest buffer by mixing in cysteine-hydrochloride to 0.01 M and papain to 0.125 mg/mL into base digest buffer (0.10 M sodium phosphate dibasic, 0.01 M Ethylenediaminetetraacetic acid disodium salt dihydrate, pH 6.5). Digest cell pellet in 200-500 μL of papain digest buffer at 60° C. for 12-18 hours. Vortex digestion mixture to dissipate digested pellet. Assay sGAG and total collagen according to known protocols. Collagen assay was modified from Cissell et al. Specifically, the reaction volume of Cissell was halved, which results in halving of input sample volume and volume of all solutions.

Sulfated glycosaminoglycans were assayed using methods derived from (Eur Cell Mater, 2015 Apr. 19; 29:224-36) with the exception that volume of DMMB solution was made to 800 mL rather than 1 L to generate a 1.25× solution. The pH of this solution was adjusted to 1.5 on the day of use with HCl and solution concentration was brought to 1× with 0.03162 M HCl to maintain 1.5 pH.

### T-Cell Immunomodulation Assay

Samples of discogenic cells from liquid nitrogen were thawed and transferred to a centrifuge tube containing 3-8 mL of DMEM/F-12 with 10-20% FBS and 25-50 ug/mL gentamicin sulfate (discogenic cell culture media). Cells were centrifuged at 200-400×g for 5 minutes to pellet the cells. The supernatant was aspirated and the cells resuspended in 1-3 mL of discogenic culture media. Cell concentration was measured using a K2 or cellaca cellometer and cell concentration adjusted to 0.2-2*10\hat{ }6 viable cells/mL accordingly. 200-300 μL of each cell suspension was added per well of a 96-well tissue culture plate (flat bottom). Plates were incubated in a cell culture incubator (37° C., 5% CO2) for 1-4 days.

After incubation, PBMCs were thawed and plated in the same plate as discogenic cells according to the following protocol. Discogenic cell culture media with mitomycin at a concentration of 30-50 μg/mL was prepared and may be referred to as ‘preculture media,’ which may be useful in inhibiting or preventing proliferation. Add 100-200 μL of preculture media with mitomycin to discogenic cells and incubate for 1.5-3 hrs at 37 C. While cells are incubating in mitomycin PBMCs are prepared as follows. PBMC samples from liquid nitrogen were thawed and transferred to a centrifuge tube containing 3-8 mL of Immunocult XF T-cell expansion media (Stem Cell Technologies) with 10-20% FBS and 25-50 ug/mL gentamicin sulfate (quenching media). Tubes containing thawed PBMCs were then centrifuged at 200-400×g for 5 minutes to pellet the cells. Supernatant was aspirated and samples resuspended in 1-3 mL of PBS. Cell concentration was measured using a K2 or cellaca cellometer and cell concentration adjusted to 1-2*10\hat{ }6 viable cells/mL accordingly. CFSE dye was resuspended in 18-40 μL of DMSO and then 0.5-1.5 μL of resuspended CFSE was added to cells, and incubated for 5-25 minutes, with periodic mixing every 5-10 minutes by vortexing. After CFSE staining was complete, 3×-4× volume of quenching media was added and the mixture incubated for 3-8 minutes. PBMC sample(s) were centrifuged at 200-400×g for 5 minutes to pellet the cells. Supernatant was aspirated and cells resuspended in appropriate amount of Immunocult XF T-cell expansion media with 10-20 ng/mL of IL-2 and 25-50 μL of gentamicin to obtain a PBMC concentration of 1-5 million cells/mL. A subset of PBMCs were activated with CD3/CD28 activator by adding activator to cells at a concentration of 2-50 μL/mL. A subset of PBMCs that were not treated with activator acted as a not activated control. Activated PBMCs were added to wells with and without discogenic cells at a density of 100,000-500,000 cells/well in 100-200 μL media. Not activated control PBMCs were added to wells without discogenic cells at the same densities.

Cell cultures were incubated for 3-5 days and then cells prepared for flow cytometry to assess PBMC proliferation according to the following steps. 100-200 μL of TRYPLE (Thermofisher) was added to each well and incubated at 37° C. until discogenic cells detach (5-10 minutes). Cells were pipetted to mix and break up clusters and transferred to a 96-well polypropylene plate. Cell plates were centrifuged for 5 minutes at 200-400×g. Supernatant was decanted and cells resuspend in 90 μL of flow buffer (PBS+1% HSA) with 0.02-0.08 ug/mL human Fc block (BD Biosciences). Resuspended cells were incubated for 10-15 minutes at RT before addition of 5-20 μL of CD4 antibody or IgG isotype control (for control samples), samples were then mixed, and stained for 30-60 minutes at 4 C. Flow buffer was added to bring the total volume to 250-300 μL and then centrifuged at 200-400×g for 5 minutes. Supernatant was decanted and 250-300 μL of flow buffer was added before centrifuging at 200-400×g for 5 minutes. Supernatant was again decanted and the cells resuspend in 200-300 μL of flow buffer, samples transferred to microcentrifuge tubes and FITC signal in CD4 positive cells was analyzed by flow cytometry. Average FITC signal of CD4+activated PBMCs without discogenic cells was calculated and this value used to calculate relative proliferation of activated CD4+PBMCs cultured with discogenic cells and not activated CD4+PBMCs using the following equation:

Relative Proliferation=100−100*((1/(FITC-M_sample))/(1/(FITC-A_control))

FITC-M sample=mean FITC signal of all CD4+ cells measured in each sample
FITC-A_control=Average FITC_M signal of all samples of activated PBMCs cultured without discogenic cells

### Statistical Analysis

For each set of experimental conditions, averages and Standard Deviation (SD) or Standard Error were calculated. When comparing multiple groups, a standard least squares regression model was created and a pairwise comparison of experimental groups was conducted by post hoc least squares means differences student's t tests at alpha=0.05.

### Computation Fluid Dynamics Methods

Detailed models of two systems (0.25 L DASbox by Eppendorf and HyPerforma 50 Liter by Thermo Fisher Scientific) were created using Sign Modeller, Meshing, Fluent & CFD Post by ANSYS 18.1. Moving reference zones were created around the impeller blades to account for the blade movement. Then, computational domains were divided into multiple minute volumes, or elements, to create a ‘mesh’ for the two tank scales. The dimensions of the elements were created small enough to capture the main features of the process being modelled, particularly in areas of strong turbulence (i.e., close to the impeller blades). After creating an initial crude mesh, the mesh was refined around the impeller blades and probes, where applicable. Initially there were an average of ˜90,000 elements in the crude mesh for each reactor. Upon refinement the number rose to 4.4 million elements. The conservation equations of transport phenomena were then applied and solved for each element. This process was carried out for each vessel configuration at seven agitation speeds each.

A set of assumptions and equations were used to create CFD models. A table of abbreviated terms is provided below. In most embodiments, the Reynolds Number (a unitless coefficient which describes the flow characteristics) is maintained in the turbulent flow region i.e. Reynolds Number >2500 (Turbulent).

| Abbreviation | Abbreviated Terms in Supplemental Materials |
|---|---|
| D | impeller diameter (m) |
| kg | Kilograms |
| L | Liter |
| M | moment around impeller (N.m) |
| mL | Milliliter |
| m | Meter |
| N | impeller speed (s⁻¹) |
| Po | Power Number |
| P | Power (watts) |
| Re | Reynolds Number |
| s | Seconds |
| s⁻¹ | Reciprocal seconds |
| V | Volume (m³) |
| ρ | fluid density (kg.m⁻³) |
| μ | liquid viscosity (kg/m/s) |
| γ | average shear rate (s⁻¹) |
| γ_{imp,tur} | impeller averaged turbulent average shear rate (s⁻¹) |

The Reynolds Number (a unitless coefficient which describes the flow characteristics) was assumed to be in the turbulent flow region i.e. Reynolds Number >2500 (Turbulent). Reynold number was calculated using the equation:

$$Re = \frac{ρ {ND}^{2}}{μ}$$

The power input can be calculated in CFD via:

P=πNM

The impeller power number, Po (−) can then be calculated:

$$P o = \frac{P}{ρ {N}^{3} {D}^{5}}$$

The shear rate calculations specifically relate to the energy dissipation in the vessel. As the energy introduced by the impeller is dissipated, this energy is consumed by the nearby fluid and other particles (specifically in this case, proteins) which can be negatively impacted by a high rate of energy dissipation (i.e. high shear). The shear rate calculations are computed as follows:

### Vessel Averaged Turbulent Shear Rate:

$$γ = {( \frac{P}{V} \frac{1}{u} )}^{0.5}$$

### Impeller Averaged Turbulent Shear Rate:

$${γ}_{imp , tur} = {( 0.3 \frac{P}{{V}_{imp}} \frac{1}{μ} )}^{0.5}$$

### Where:

$${V}_{imp} = ( \frac{{D}^{2}}{4} π \frac{D}{4} )$$

### Selection of New Culture Modality

Cells were grown in static suspension culture, wave reactors, waterwheels, Erlenmeyer shake flasks, and 0.25 L STR. The cells grown in the static culture generated typical Discogenic Cell spheres (FIG. 1 Panel A). Cells grown in the waterwheels (1.16× relative doublings, SD 0.04) formed into long sheets of cells which trailed the wheel and built up in the vessel corners (FIG. 1 Panel B). Cells grown in Erlenmeyer shake flasks had similar doublings relative to the original static culture controls (100 RPM: 1.04× relative doublings, SD 0.09; 130 RPM: 1.07× relative doublings, SD 0.32), but spheres were not generated and cells attached to the vessel walls and formed into clumps (FIG. 1 Panel C). Cell clumping was most egregious in the wave reactor where large rafts of cells formed (0.50× relative doublings, SD 0.31) (FIG. 1 Panel D).

In the 0.25 L STR, cells grown at the low RPM setting formed spheres. However, spheres grew too large in size (FIG. 1 Panel E) causing issues with spheres settling out of solution (FIG. 1 Panel F); the medium and high RPM settings resulted in limited sphere formation (FIG. 1 Panel G). Doublings were consistently higher in the STR than in the original static suspension culture (Low RPM: 1.69× relative doublings, SD 0.42; medium RPM: 2.51× relative doublings, SD 0.00; high RPM: 2.19× relative doublings, SD 0.18). Because spheres formed at the low and medium RPM in the 0.25 L STR, relative doublings were twice that of static suspension culture controls, and due to the potential for scalability, the STR modality was chosen for further optimization.

### Agitation Modeling Study

Next, an experiment employing 22 DASbox was run to evaluate different agitation strategies. No reactors run at a single RPM for the duration of culture enabled sphere growth while simultaneously limiting attachment to vessel walls. In contrast, when RPM was ramped over the course of the culture, we saw that small spheres successfully formed at the beginning of the run and then grow without settling. The ramped agitation allowed for sphere formation which visually resembled spheres from our static culture modality (FIG. 1 Panel H).

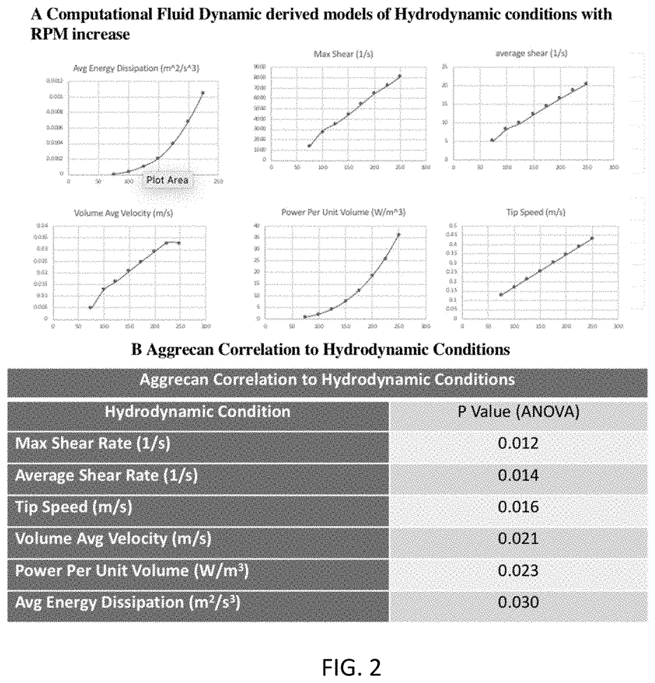

The data generated from these runs were used for CFD modeling of 8 different agitation rates. Hydrodynamic parameters were found to vary differently as a function of RPM (FIG. 2 Panel A). At the same scale but different RPM, average energy dissipation (m²/s³) and power per unit volume (W/m³) resulted in exponential curves whereas maximum shear rate (1/s), average shear rate (1/s), volume average velocity (m/s) and tip speed (m/s) had linear slopes. In each case the slope was different, indicating that the various hydrodynamic conditions do not scale equally based on RPM. By modeling aggrecan expression from the 22 DASbox reactors with the various hydrodynamic conditions, we found that maximum shear rate (1/s) had the highest correlation with aggrecan expression (p=0.012) (FIG. 2 Panel B).

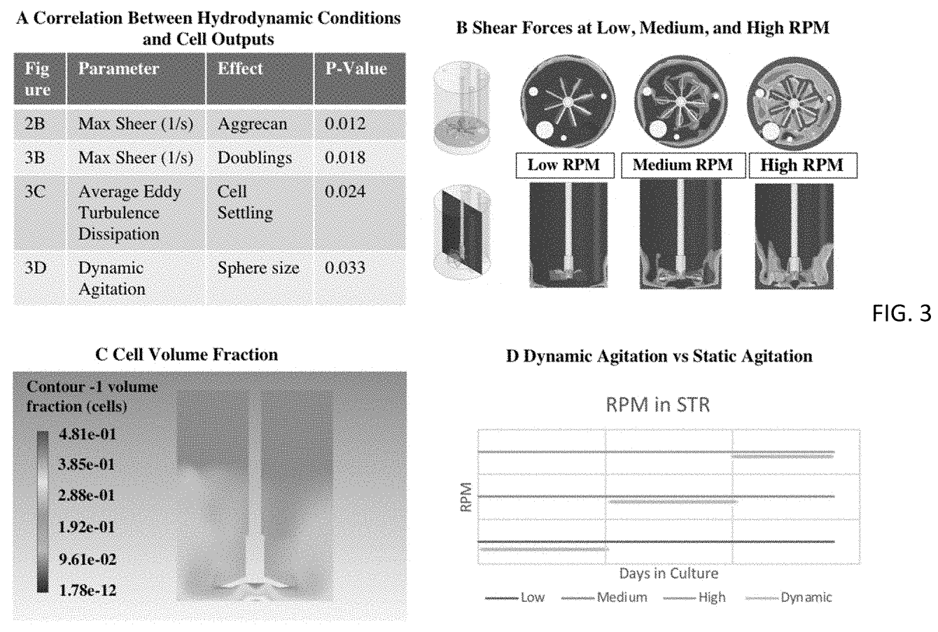

The CFD model of various agitation profiles showed us which metrics impact cell growth in STR (FIG. 3 Panel A). As previously described, max shear rate (1/s) impacted aggrecan production (p=0.012), along with cell doublings (p=0.018) (FIG. 3 Panel B). Also, cell settling was impacted by the cell volume fraction, meaning cell spheres would fall out of solution as predicted by average eddy turbulence dissipation (p=0.024) (FIG. 3 Panel C). Ultimately, sphere size was impacted by the use of dynamic agitation, where the use of a ramping vs static agitation scheme significantly correlated with on sphere sizes (p=0.033) (FIG. 3 Panel D). With these findings, new set-points were identified for the DASbox which we predicted would produce Discogenic Cells with comparable properties to cells grown in static culture.

### Process Tunability Modeling

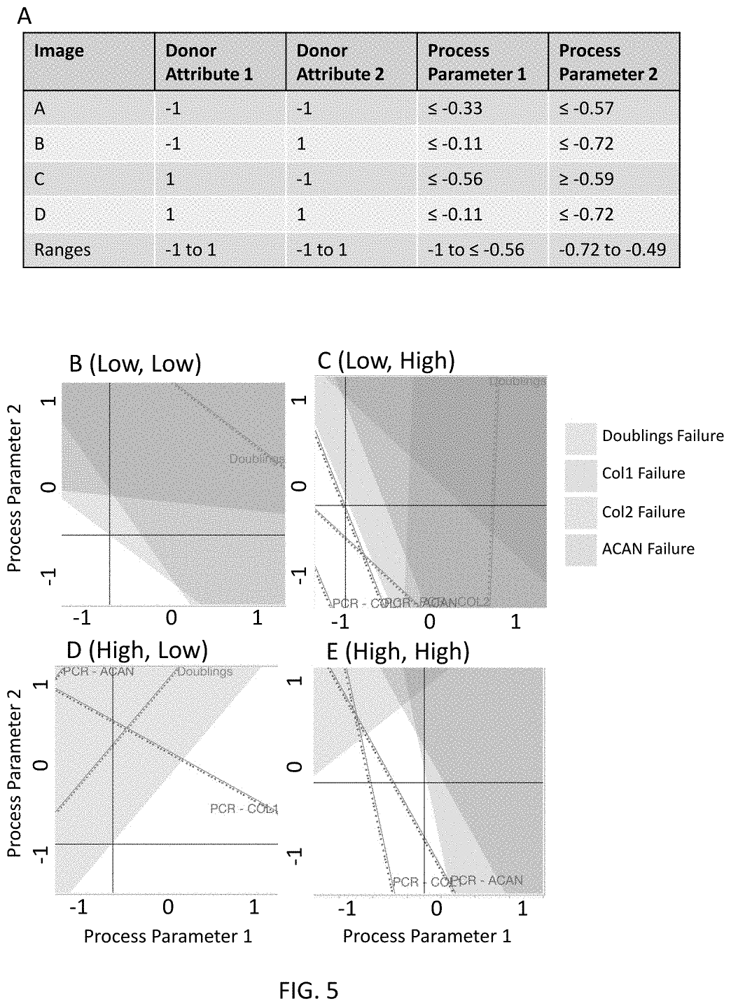

The relationship between cell doublings or potency (as reflected in biomarkers levels measured by various methods, for example via PCR) were modeled for input factors including donor attributes, media attributes and process parameters was established. These models determined that 5 of the 11 measured input factors impact doublings and 11 of 11 factors impact PCR potency. Using regression models and a series of two-factor contour profilers, the acceptable ranges of process parameters for two high-risk donor attributes and two high-risk process parameters are defined (FIG. 5). Analysis of each combination of the highest and lowest donor attribute values, provided insight into the impact of donor variation. Applicants have used these insights to develop methods, protocols, and systems to obtain cell populations with desired performance attributes from donors with a wide variety of starting attributes. In some embodiments, varying media and process parameters to arrive at cells with specific (or a range of) performance attributes may be referred to as ‘tunability.’

FIG. 5 shows results from the present studies. Shaded areas in the contour profiler show failure conditions for each of the quality metrics. Ranges which are unshaded across all conditions indicate acceptable operating ranges.

Cell doublings were recorded and found to be normally-distributed using an Anderson-Darling Goodness-of-Fit Test (p>0.079). Despite conducting all STR runs within ranges previously established using one factor at a time experimentation, our multi-variate experimentation conditions resulted in 44% of all STR runs with zero cell doublings and in 84% failing to meet process doubling requirements. The failure of previous univariate analysis to capture 84% of failure conditions establishes the necessity for multi-variate experimentation when characterizing a bioprocess across multiple allogeneic cell lines. The initial agitation speed may be selected to provide for both maintenance of the cells in suspension and allowing formation of cell spheres—that is, avoidance of forces sufficient to separate or tear apart cell spheres while preventing or reducing the ability of cells and spheres to settle out of solution.

Comparison of Static vs. STR Modality

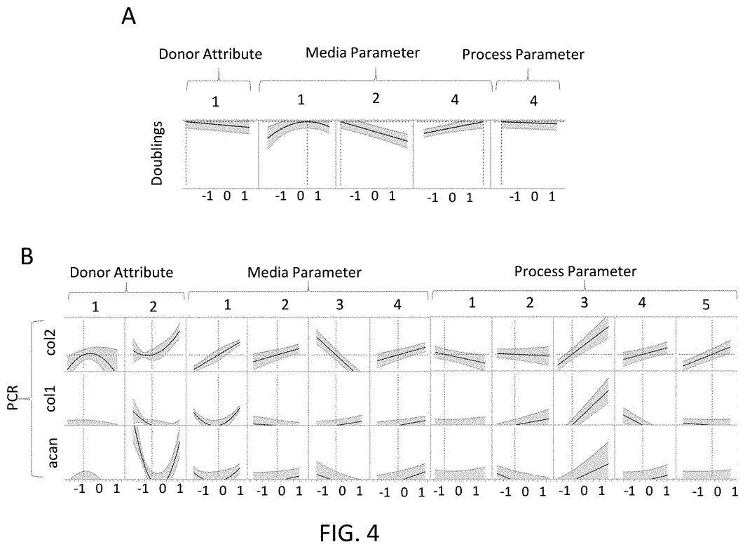

The agitation ramp profile that demonstrated the ability to generate spheres with minimal settling or sticking was carried forward and used for subsequent split stream growth between 0.25 L STR and static suspension culture (FIG. 4A). In both modalities, all the cells formed the characteristic spheres (FIG. 4B). Sphere size, aggrecan expression, and cell doublings did not exhibit statistically significant differences between the new STR process and the original static suspension method. (FIG. 4C). Flow cytometry showed no significant differences between the two modalities (FIG. 4D).

Cells from both modalities were then tested in an in vivo rabbit model of disc degeneration. Following the initial injury, x-ray analysis showed that the disc height index decreased by 25-50%. After dosing (FIG. 5A), the disc height was restored slightly in the vehicle groups, and more substantially in the cell therapy groups (p=0.019) (FIG. 5B). Cells grown using either process had significantly higher disc height than sham after 6 weeks in vivo (p>0.05).

Histological analysis by a pathologist identified abnormally high density and cellularity in the nucleus pulposus from discs that received sham or vehicle, hypothesized to be created by the loss of hydration caused by needle puncture injury and subsequent degeneration. In contrast, the discs injected with cell therapy had a more normal appearance in these parameters (FIG. 5C). Dorsal cartilage and osteophyte formation were noted but unassociated with treatment condition and are attributed to the injury model. No inflammation or toxicity was noted that may have been caused by the cell therapy. Also, the new vehicle (which contains the original excipients minus one in different concentrations) did not result in any abnormal findings. Histologically, no abnormal tissue (fat, bone, etc.) was noted in the discs with cell therapy that could account for the structural increase in disc height measured via x-ray.

Scale-Up into Large-Scale STR

The cell volume fraction that was modeled indicated that a scale-up strategy based on tip speed (m/s) or average energy dissipation (m²/s³) would result in the majority of the cells settling out of solution. Also, scale-up using average shear (1/s) would result in an extremely high RPM that would prevent sphere formation and promote cell death. Scale-up based on power per unit volume (W/m³), maximum shear rate (1/s), and volume average velocity (m/s) all should limit cell settling while allowing sphere formation. Of these hydrodynamic conditions, maximum shear rate (1/s) was chosen to scale into the 50 L because it had the strongest correlation with aggrecan expression.

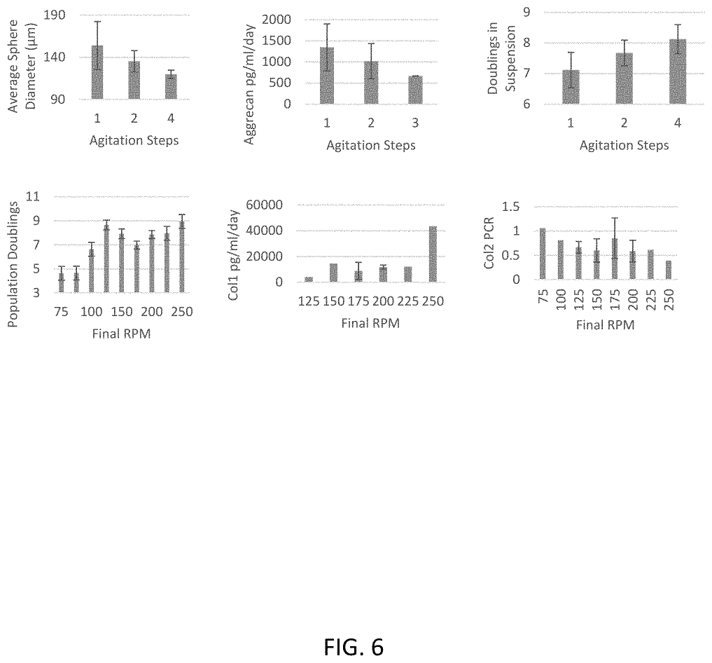

Cells were split and grown in 0.25 L and 50 L STRs (FIG. 6A). Maximum shear rate (1/s) was maintained between scales. Spheres formed successfully at both the 0.25 L and 50 L scale (FIG. 6B). The 50 L had comparable doublings (1.00 doublings relative to DASbox mean) (FIG. 6C). Sphere size was also comparable between the two DASbox (relative sphere size 1.00, SD 0.07) and the 50 L (relative sphere size DASbox 0.98, SD 0.44). Lastly, flow cytometry found no notable differences in surface markers (FIG. 6D).

### Analysis of Discogenic Cells

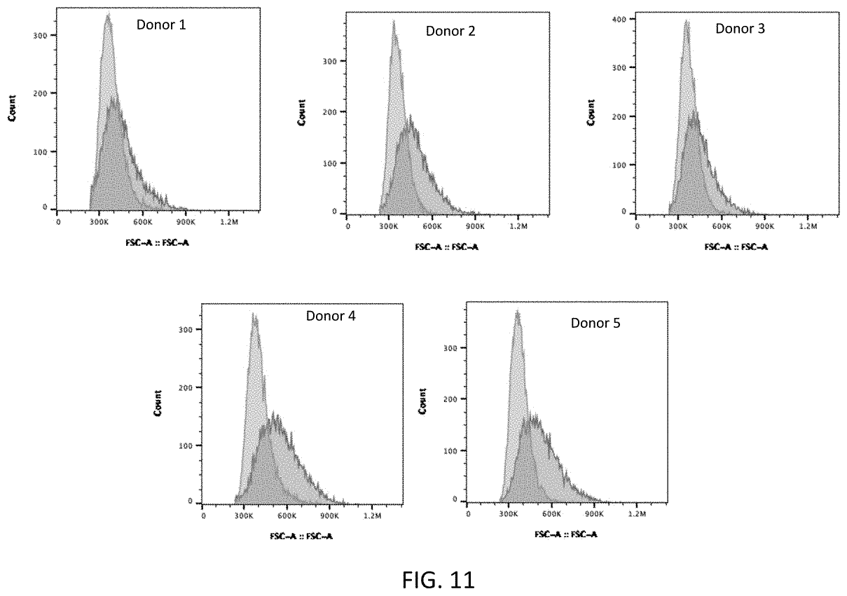

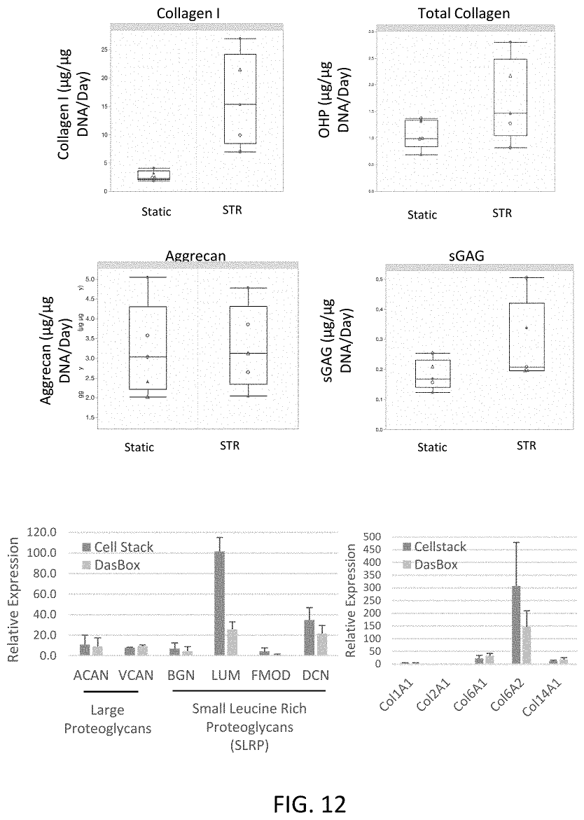

When cells from five distinct donors were grown in both static flask culture and STRs, the resulting histograms of forward scatter were different between the methods. Notably, the cells generated in STRs were more uniform in size, and smaller (FIG. 11). Also, while differences were noted in Collagen 1 production between the two modalities, no differences were noted in total collagen production (FIG. 12). Neither aggrecan nor sGAG showed differences between the modalities (FIG. 12). When other ECM molecules were evaluated, both lumican and collagen 6A2 showed significant expression and differences between modalities (FIG. 12).

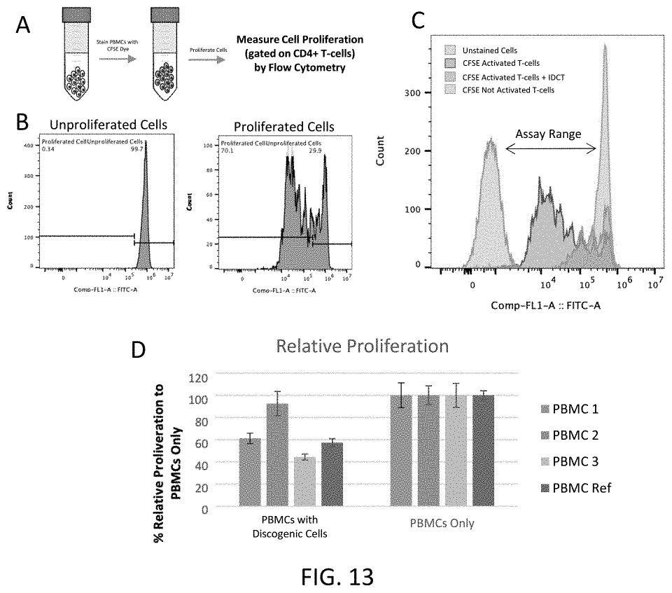

The cells not only produce extracellular matrix, but also have anti-inflammatory properties as measured using a CSFE-dye incorporation method via flow cytometry (FIG. 13). Proliferation of activated PBMCs (blood cells) is suppressed in the presence of discogenic cells, show an immunomodulatory effect that could be beneficial for treating disc degeneration.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed description is to be regarded as illustrative in nature and not restrictive.

All references disclosed herein, whether patent or non-patent, are hereby incorporated by reference as if each was included at its citation, in its entirety. In case of conflict between reference and specification, the present specification, including definitions, will control.

Although the present disclosure has been described with a certain degree of particularity, it is understood the disclosure has been made by way of example, and changes in detail or structure may be made without departing from the spirit of the disclosure as defined in the appended claims.

We claim:

1. A method for culturing mammalian cells comprising:
collecting a plurality of mammalian cells;
introducing the plurality of mammalian cells into a container or vessel, the container or vessel comprising a sphere culture media to create a cell/media mixture;
agitating the cell/media mixture at an agitation speed corresponding to a first energy level, wherein the first energy level is sufficient to maintain greater than 90% of the cells in suspension;
allowing the plurality of cells to grow, divide, and form cell spheres of a first diameter;
increasing the agitation speed to a second energy level, wherein the second energy level is higher than the first energy level and sufficient to maintain the cell spheres in suspension;
maintaining the agitation speed to allow the cell spheres to achieve a greater diameter while remaining in suspension.

2. The method of claim 1, wherein the mammalian cell is selected from a progenitor cell, stem cell, or pluripotent cell.

3. The method of claim 1 or 2, wherein the mammalian cell is derived from muscle, brain, spinal cord, peripheral nerve, kidney, eye, skin, blood vessel, hair follicle, amnion, chorion, umbilical cord, placenta, liver, heart, lung, pancreas, cartilage, bone, thymus, thyroid, blood, lymph node, cartilage, and intervertebral disc cells.

4. The method of any one of claims 1-3, wherein the mammalian cell are cartilage cells.

5. The method of any one of claims 1-3, wherein the mammalian cells are intervertebral disc cells.

6. The method of any one of claims 1-5, where the increasing the agitation speed to a second energy level is linear over time.

7. The method of any one of claims 1-5, wherein the increasing the agitation speed to a second energy level is non-linear.

8. The method of claim 7, wherein the increasing the agitation speed to a second energy level includes at least one step.

9. The method of any one of claims 1-8, wherein the sphere culture media lacks a scaffold molecule.

10. The method of any one of claims 1-9, wherein the plurality of cells is grown in attachment culture prior to introducing the plurality of mammalian cells into a container or vessel comprising the sphere culture media.

11. The method of claim 10, wherein the cells attached to the solid surface are allowed to double prior to introducing the plurality of mammalian cells into a container or vessel, the container or vessel comprising a sphere culture media.

12. A method for culturing a mammalian cell population in dynamic suspension, comprising:
introducing the mammalian cell population into a bioreactor containing sphere culture media on a first day to create a cell/media mixture;
agitating the cell/media mixture at a first agitation speed;
allowing the cell population in the cell/media mixture to form cell spheres;
agitating the cell/media mixture at a second speed, wherein the difference between the first speed produces a first shear value that is less than a first max shear value, and the second speed produces a second shear value that is less than a second max shear value.

13. A method for dynamic culturing of mammalian cell spheres:
introducing a mammalian cell population into a bioreactor containing sphere culture media to create a cell/media mixture;
agitating the cell/media mixture at a first agitation speed sufficient to prevent or inhibit the cells exiting suspension;
allowing the cell population in the cell/media mixture to form cell spheres;
maintaining the majority of cells in suspension;
isolating and collecting the suspended cell spheres; and thereby dynamically culturing mammalian cell spheres.

14. A method of modifying one or more characteristics of a therapeutic mammalian cell population comprising:
isolating a population of cells from a donor tissue, wherein the donor has a first attribute with a first attribute score and a second attribute with a second attribute score;
determining a desired characteristic of for the therapeutic cell population;
selecting a first media parameter based on the first attribute score and/or the second attribute score;
selecting a process parameter based on the first attribute score and/or the second attribute score and/or the media parameter;
culturing the population of cells in suspension in a container or vessel comprising a sphere culture media;
maintaining the population of cells in suspension;
allowing the cells to divide and grow to form clonal cell spheres;
maintaining the population of cell spheres in suspension;
isolating and collecting suspended cell spheres having the pre-determined characteristic; and thereby
modifying one or more characteristics of a therapeutic mammalian cell population.

15. The method of any one of claims 12-14, wherein the mammalian cell is selected from a progenitor cell, stem cell, or pluripotent cell.

16. The method of any one of claims 12-15, wherein the mammalian cell is derived from muscle, liver, heart, lung, pancreas, bone, thyroid, blood, lymph node, brain, spinal cord, peripheral nerve, kidney, eye, skin, blood vessel, hair follicle, amnion, chorion, cartilage, intervertebral disc cells, umbilical cord, and placenta.

17. The method of any one of claims 12-16, wherein the mammalian cell are cartilage cells.

18. The method of any one of claims 12-17, wherein the mammalian cells are intervertebral disc cells.

19. The method of any one of claims 12-18, where the increasing the agitation speed to a second energy level is linear over time.

20. The method of any one of claims 12-18, wherein the increasing the agitation speed to a second energy level is non-linear.

21. The method of claim 20, wherein the increasing the agitation speed to a second energy level includes at least one step.

22. The method of any one of claims 12-21, wherein the sphere culture media lacks a scaffold molecule.

23. The method of any one of claims 12-22, wherein the plurality of cells are grown in attachment culture prior to introducing the plurality of mammalian cells into a container or vessel comprising the sphere culture media.

24. The method of claim 23, wherein the cells attached to the solid surface are allowed to double prior to introducing the plurality of mammalian cells into a container or vessel, the container or vessel comprising a sphere culture media.

25. A mammalian cell population comprising:
intervertebral disc cells, wherein greater than 90% of the cells are negative for a surface marker selected from CD24, HLA-DR/DP/DQ, CD45, CD40, CD271, CD80, CD86, or a combination thereof;
positive for a surface marker selected from CD44, CD73, CD90, HLA-ABC or a combination thereof.

26. A mammalian cell population comprising:
intervertebral disc cells, wherein greater than 90% of the cells are express one or more of aggrecan, collagen 1, collagen 2, collagen 6, collagen 14, decorin (DCN), biglycan (BGN), lumican (LUM), and fibromodulin (FMOD); anti-inflammatory effect as seen through activated T-cell assays.

27. A method for culturing mammalian cells comprising:
collecting a plurality of mammalian cells;
introducing the plurality of mammalian cells into a container or vessel, the container or vessel comprising a sphere culture media to create a cell/media mixture;
agitating the cell/media mixture at an agitation speed corresponding to a first energy level, wherein the first energy level is sufficient to maintain greater than 90% of the cells in suspension;
allowing the plurality of cells to grow, divide, and form cell spheres of a first diameter;
maintaining the agitation speed to allow the cell spheres to achieve a greater diameter while remaining in suspension.

28. The method of claim 27, wherein the mammalian cell is selected from a progenitor cell, stem cell, or pluripotent cell.

29. The method of claim 27 or 28, wherein the mammalian cell is derived from muscle, brain, spinal cord, peripheral nerve, kidney, eye, skin, blood vessel, hair follicle, amnion, chorion, umbilical cord, placenta, liver, heart, lung, pancreas, cartilage, bone, thymus, thyroid, blood, lymph node, cartilage, and intervertebral disc cells.

30. The method of any one of claims 27-29, wherein the mammalian cell are cartilage cells.

31. The method of any one of claims 27-30, wherein the mammalian cells are intervertebral disc cells.

32. The method of any one of claims 27-31, wherein the sphere culture media lacks a scaffold molecule.

33. The method of any one of claims 27-32, wherein the plurality of cells is grown in attachment culture prior to introducing the plurality of mammalian cells into a container or vessel comprising the sphere culture media.

34. The method of any one of claims 27-33, wherein the cells attached to the solid surface are allowed to double prior to introducing the plurality of mammalian cells into a container or vessel, the container or vessel comprising a sphere culture media.

35. The method of any one of claims 27-34, comprising increasing the agitation speed to a second energy level, wherein the second energy level is higher than the first energy level and sufficient to maintain the cell spheres in suspension.

36. The method of any one of claim 35, where the increasing the agitation speed to a second energy level is linear over time.

33. The method of any one of claims 35-36, wherein the increasing the agitation speed to a second energy level is non-linear.

34. The method of claim 33, wherein the increasing the agitation speed to a second energy level includes at least one step.
